(19) United States
(12) Patent Application Publication
(10) Pub. No.: US 2019/0316116 A1
(43) Pub. Date: Oct. 17, 2019

(54) HIGHLY STABLE POLYPEPTIDE SCAFFOLDS

(71) Applicant: MONASH UNIVERSITY, Clayton, Victoria (AU)

(72) Inventors: Benjamin POREBSKI, Cambridge (GB); Ashley BUCKLE, Mulgrave (AU)

(73) Assignee: MONASH UNIVERSITY, Clayton, Victoria (AU)

(21) Appl. No.: 15/535,899

(22) PCT Filed: Dec. 15, 2015
(86) PCT No.: PCT/AU2015/050795

§ 371 Date: Jun. 14, 2017

(30) Foreign Application Priority Data
Dec. 15, 2014 (AU) 2014905069

(51) Int. Cl. C12N 15/10; C07K 14/78

(52) U.S. Cl. CPC C12N 15/1044; C07K 14/78

(57) ABSTRACT

The invention relates to a highly stable polypeptide scaffold comprising an amino acid sequence based on a fibronectin type III (Fn3) domain and a polypeptide comprising the scaffold with grafted loops. The invention also relates to a nucleic acid molecule encoding the polypeptide scaffold, a method of making the polypeptide scaffold, and a composition comprising the polypeptide scaffold.

### CROSS-REFERENCE TO PRIOR APPLICATIONS

This application is a § 371 National Stage Application of PCT International Application No. PCT/AU2015/050795 filed Dec. 15, 2015, which claims benefit to Australian Patent Application No. 2014905069 filed Dec. 15, 2014, both of which are herein incorporated by reference in their entirety.

### REFERENCE TO A SEQUENCE LISTING

The instant application contains a sequence listing which has been submitted in ASCII format via EFS-Web and is hereby incorporated by reference in its entirety. Said ASCII copy, created on Jun. 8, 2017, is named 217412_0001_sequencelisting.txt and is 13,606 bytes in size.

### FIELD

The invention relates to the field of antibody mimetics and more particularly to highly stable polypeptide scaffolds based on fibronectin and uses thereof.

### BACKGROUND

Monoclonal antibodies are therapeutic proteins with high affinity and specificity for a target. They contain one or more regions which are amenable to specific or random sequence variation to enable the binding of the antibody to a particular target with a particular specificity. Non-antibody proteins that can be engineered to bind such targets are also of high interest in the biopharmaceutical industry. These “alternative scaffold” proteins offer advantages over traditional antibodies due to their small size, lack of disulfide bonds, improved stability, and ability to be expressed in prokaryotic hosts. Novel methods of purification are readily applied; they are easily conjugated to drugs/toxins, penetrate efficiently into tissues and are readily formatted into multi-specific binders.

One type of alternative scaffold comprises an immunoglobulin (Ig) fold. This fold is found in the variable regions of antibodies as well as in thousands of non-antibody proteins. One protein comprising an Ig fold is fibronectin type III (Fn3), a domain found widely across phyla and protein classes, such as in mammalian blood and structural proteins. The Fn3 domain occurs often in various proteins, including fibronectins, tenascins, intracellular cytoskeletal proteins, cytokine receptors and prokaryotic enzymes. Fn3 domains comprise seven beta strands, designated N-terminus to C-terminus A, B, C, D, E, F, and G strands, each strand separated by a loop region wherein the loop regions are designated N-terminus to C-terminus, AB, BC, CD, DE, EF, and FG loops. This structure is reminiscent of natural or engineered antibodies and contains loops that are analogously located to the complementarity determining regions (CDRs) of an antibody variable domain.

It has been shown that the CDR-like loop regions of fibronectin based scaffolds can tolerate a number of mutations in the surface exposed loops while retaining the overall Ig fold structure. Libraries of amino acid variants have been built into these loops and specific binders selected to a number of different targets.

There is a need to develop improved polypeptide scaffolds, having increased stability, for a variety of therapeutic and diagnostic applications. The present invention seeks to provide highly stable polypeptide scaffolds based on the Fn3 domain, which scaffolds may be used in the production of libraries for screening for therapeutic and diagnostic agents.

### SUMMARY

A first aspect provides a highly stable polypeptide scaffold comprising an amino acid sequence based on a fibronectin type III (Fn3) domain, in which the amino acid sequence comprises at least:

### (A) Y44, Y48, Y67 and Y78;

(B) L20 and V96 and/or V22 and V98; and
(C) R45 and one, two, three, four, five or all of R49, R81, R83, E47, E57 and E79, wherein amino acids are numbered according to their position in SEQ ID NO: 1.

(SEQ ID NO: 1)
X₁₋₁₃ XXXXXX LXVXXXXXXX XXXXXXXXXX XXXXYRXEYR
XXXXXXXEXX XXXXXXXYXX XXXXXXXXYE XRXRXXXXXX
EXXXXXVXVXX

The polypeptide scaffold amino acid sequence may further comprise E90. In the event of loop randomisation this residue is likely to be changed as it sits in the FG loop.

Without wishing to be bound by theory, the inventors propose that the combination of tyrosine residues in Group A are important to the stability of the polypeptide scaffold via mediation of interactions with water, thus protecting the hydrophobic core.

Without wishing to be bound by theory, the inventors propose that the pairs of hydrophobic residues in Group B and particularly the combination of both pairs of hydrophobic residues in Group B are important to the stability of the polypeptide scaffold by providing favourable interactions amongst the N and C terminal strands of the molecule at elevated temperatures.

Without wishing to be bound by theory, the inventors propose that one or more of the electrostatically charged residues in Group C and particularly the combination of electrostatically charged residues in Group C is important to the stability of the polypeptide scaffold by providing a complementary salt bridge network on the surface of β-sheet 2. This is likely to restrict unfolding of the molecule at high temperatures.

Group B comprises amino acid residues L20 and V22 or V96 and V98 or L20 and V22 and V96 and V98.

Group C comprises:

### R45 and R49,

### R45 and R81,

### R45 and R83,

### R45 and E47,

### R45 and E57,

### R45 and E79,

### R45, R49 and R81,

### R45, R49 and R83,

### R45, R49 and E47,

### R45, R49 and E57,

### R45, R49 and E79,

### R45, R81 and R83,

### R45, R81 and E47,

### R45, R81 and E57,

### R45, R81 and E79,

### R45, R83 and E47,

### R45, R83 and E57,

### R45, R83 and E79,

### R45, E47 and E57,

### R45, E47 and E79,

### R45, E57 and E79,

### R45, R49, R81 and R83,

### R45, R49, R81 and E47,

### R45, R49, R81 and E57,

### R45, R49, R81 and E79,

### R45, R49, R83 and E47,

### R45, R49, R83 and E57,

### R45, R49, R83 and E79,

### R45, R49, E47 and E57,

### R45, R49, E47 and E79,

### R45, R49, E57 and E79,

### R45, R81, R83 and E47,

### R45, R81, R83 and E57,

### R45, R81, R83 and E79,

### R45, R83, E47 and E57,

### R45, R83, E47 and E79,

### R45, R81, E47 and E57,

### R45, R81, E57 and E79,

### R45, R49, R81, R83 and E47,

### R45, R49, R81, R83 and E57,

### R45, R49, R81, R83 and E79,

### R45, R81, R83, E47 and E57,

### R45, R81, R83, E47 and E79,

### R45, R83, E47, E57 and E79,

### R45, R49, R81, R83, E47 and E57,

### R45, R49, R81, R83, E47, and E79,

### R45, R81, R83, E47, E57 and E79,

### R45, R49, R83, E47, E57 and E79,

### R45, R49, R81, E47, E57 and E79,

### R45, R49, R81, R83, E57 and E79,

### R45, R49, R81, R83, E47, E57 and E79.

In one embodiment the polypeptide scaffold comprises residues 14-98 of the amino acid sequence of SEQ ID NO: 1, in which X represents any amino acid residue.

(SEQ ID NO: 1)
X₁₋₁₃ XXXXXX LXVXXXXXXX XXXXXXXXXX XXXXYRXEYR
XXXXXXXEXX XXXXXXXYXX XXXXXXXXYE XRXRXXXXXX
EXXXXXVXVXX

In a preferred embodiment, the protein scaffold comprises residues 14-98 the amino acid sequence of SEQ ID NO: 2, referred to hereinafter as Fn3con or a variant thereof, with non-essential residues shown in lower case.

### FN3 con

X₁₋₁₃ psppgn LrVtdvtsts vtlswepppg pitgYRvEYR eaggewkEvt vpgsetsYtv tglkpgteYE fRvRavngag EgppssVsVtt (SEQ ID NO:2).

Variants of SEQ ID NO: 2 include those where one or more of the lower case residues is modified by substitution, deletion or insertion with a residue that modulates immunogenicity, solubility or other functional property of the scaffold, for example one or more in vivo properties related to biodistribution, persistence in the body, or therapeutic efficacy such as the association with molecules which alter cellular, particularly, epithelial cell uptake, for example, the Fc region of an antibody, or molecules designed to bind serum proteins such as an albumin binding domain.

The loop regions of SEQ ID NO: 1 and 2 are at or about residues 26-29 for the A/B loop, 36-42 for the B/C loop, 50-54 for the C/D loop, 61-65 for the D/E loop, 70-76 for the E/F loop and 85-93 for the F/G loop. The polypeptide scaffold of the invention can be engineered by methods known in the art, including inserting residues in one or more of these loop regions, to form a binding domain selective for a binding partner. The binding partner may be a soluble molecule or a cellularly anchored molecule, for example, the extracellular domain of a receptor protein.

The polypeptide scaffolds of the invention may be used as monomeric units or linked to form polymeric structures with the same or different binding partner specificity.

The polypeptides with enhanced stability provide scaffolds with improved ease of purification, formulation, and increased shelf-life. Engineered binding partners with improved overall stability can be produced by introducing randomized peptides into loops of the stabilized scaffold.

A second aspect provides a polypeptide comprising the polypeptide scaffold of the first aspect incorporating one or more insertions or substitutions in one or more loop regions of the scaffold.

In one embodiment the insertion or substitution comprises a binding domain selective for a binding partner.

In one embodiment the insertion or substitution is a binding loop from a FN3 domain.

In one embodiment the substitution causes a deletion or insertion of residues and either lengthens or shortens the loop(s).

A third aspect provides a nucleic acid molecule encoding the polypeptide scaffold of the first aspect or the polypeptide of the second aspect.

A fourth aspect provides a nucleic acid molecule complementary to the nucleic acid molecule of the third aspect or capable of hybridising to the nucleic acid molecule of the third aspect under selected stringency conditions.

In one embodiment the complementarity is 70%, 80%, 90%, 95%, 99% or 100% across the whole nucleic acid molecule or a fragment of 20, 30, 40, 50, 60, 70, 80 or more nucleotides.

A fifth aspect provides a vector comprising the nucleic acid molecule of the third or fourth aspect.

A sixth aspect comprises a host cell comprising and optionally transformed with the nucleic acid molecule of the third or fourth aspect or with the vector of the fifth aspect.

A seventh aspect provides a method of making the stable polypeptide scaffold of the first aspect or the polypeptide of the second aspect, the method comprising culturing the host cell of the sixth aspect to produce the polypeptide scaffold or polypeptide and recovering the scaffold or polypeptide.

An eighth aspect provides a polypeptide scaffold or polypeptide produced by the method of the seventh aspect.

A ninth aspect provides at least one composition comprising the polypeptide scaffold of the first or eighth aspect or the polypeptide of the second or eighth aspect and a suitable and/or pharmaceutically acceptable carrier or diluent.

A tenth aspect provides a method of generating libraries comprising the polypeptide scaffold of the first or eighth aspects by altering the amino acid composition of a single loop or the simultaneous alteration of multiple loops or additional positions of the scaffold polypeptide of the first or eighth aspects. The loops that are altered can be lengthened or shortened accordingly. Such libraries can be generated to include all possible amino acids at each position, or a designed subset of amino acids. The library members can be used for screening by display, such as in vitro display (DNA, RNA, ribosome display, etc.), yeast, bacterial, and phage display.

An eleventh aspect provides a scaffold library made by the method of the tenth aspect.

The polypeptide scaffolds of the present invention provide enhanced biophysical properties, such as stability under conditions of high osmotic strength and solubility at high concentrations. The domains of the scaffold polypeptides are not disulfide bonded, making them capable of expression and folding in systems devoid of enzymes required for disulfide linkage formation, including prokaryotic systems, such as E. coli, and in in vitro transcription/translation systems, such as the rabbit reticulocyte lysate system.

A twelfth aspect provides a method of generating a scaffold molecule that binds to a particular target by panning the scaffold library of eleventh aspect with the target and detecting binders.

In other related aspects, the invention comprises screening methods that may be used to generate or affinity mature polypeptide scaffolds with the desired activity, e.g., capable of binding to target proteins with a certain affinity. Affinity maturation can be accomplished by iterative rounds of mutagenesis and selection using systems, such as phage display or in vitro display. Mutagenesis during this process may be the result of site directed mutagenesis to specific scaffold residues, random mutagenesis due to error-prone PCR, DNA shuffling, and/or a combination of these techniques.

### BRIEF DESCRIPTION OF THE DRAWINGS

Embodiments of the invention will now be described by way of example only with reference to the accompanying drawings in which:

FIGS. 1 A and B show the purification of FN3con. (A) An SDS page gel of the FN3con purification process, showing cell lysis, NiNTA elution fraction and the size exclusion peaks from B. (B) Size exclusion chromatography plot of FN3con from NiNTA elution, with peak 3 being FN3con.

FIGS. 2 A, B, C and D show the thermal stability, chemical stability and folding kinetics of FN3con. (A) Thermal unfolding monitored by CD at 222 nm with non-linear fit (R²=0.95); (B) Thermal unfolding in 2 M GuHCl (solid line) and FNfn8 (dashed line) is represented as fraction folded with a non-linear fit (R²=0.98 and R²=0.76 respectively); (C) Equilibrium unfolding (circles) and refolding (triangles) curves; (D) Kinetic folding data showing curvature in both arms of the chevron plot.

FIG. 3 shows reversible thermal folding of FN3con in 2 M GuHCl, monitored by CD at 222 nm. FN3con was heated from 20° C. to 110° C. (solid) and cooled from 110° C. to 20° C. (dashed). Respective non-linear fits were applied to the individual data points (R2=0.98 forwards and R2=0.98 reverse).

FIG. 4 shows a structural analysis of charged residues (sticks) within FN3con, Fibcon, FNfn10, Tencon, FNfn8 and TNfn3. Residue numbering is not included for clarity.

FIGS. 5 A, B, C, D and E show plots of physiochemical properties from Tables 3 and 4 against determined melting temperatures of the FN3 domains. (A) Number of hydrogen bonds (solid line) and salt bridges (dashed line). (B) Solvent accessible surface area of respective FN3 domains plotted against temperature. (C) The grand average hydropathy (GRAVY) score of respective FN3 domains. (D) Solvent inaccessible cavity volume of FN3 domains in respect to their melting temperatures. Lower value indicated less cavity volume. (E) Mean protein packing value (OSP), larger value indicating better surface packing.

FIGS. 6 A, B, C and D show potentially destabilizing like-charged residue clusters. (A) FNfn10 showing two separate clusters; (B) Tencon, showing E67 and E78, which are surrounded by two complementary charged clusters; (C) FNfn8, showing D26 and E75; (D) TNfn3 showing like charged residue clusters on each set of loop hairpins. The left panel shows the N-terminal loop region, highlighting potential destabilizing interactions between E33 in strand C and D49 in strand C′, as well as a potential long range repulsion from E28, D30 and D78. The right panel shows the C-terminal loop regions, highlighting potential destabilizing interactions between E9 in strand A and E86 in strand G, D15 in the A-B loop and D65 in the E-F loop, and D40 in the C-C′ loop and E67 in strand F.

FIG. 7 provides an analysis of hydrophobic residue positions in FN3 domains. A schematic unfolded model of each FN3 domain is shown, indicating positions of the hydrophobic residues as ovals. White ovals indicate the residue as contributing to the hydrophobic core and, for the most part, not solvent exposed. Grey ovals indicate exposure to solvent and lack of contribution to the hydrophobic core.

FIG. 8 shows the position of tyrosine residues in FN3con, Fibcon, FNfn10, Tencon, FNfn8 and TNfn3.

FIGS. 9 A and B show root mean square deviation (RMSD) plots at 300 K and 368 K. All plots represent the mean RMSD across replicate simulations (n=3), for Ca atoms. (A) RMSD plot of FN3con (circles), Fibcon (crosses), FNfn10 (squares), Tencon (plusses), FNfn8 (diamonds), TNfn3 (triangles) at 300 K; (B) RMSD plot of FN3con (circles), Fibcon (crosses), FNfn10 (squares), Tencon (plusses), FNfn8 (diamonds), TNfn3 (triangles) at 368 K.

FIGS. 10 A, B and C show the dynamics of C and N terminal strands at 368 K. (A) Cartoon representations of FN3con, FNfn10 and FNfn8 (grey) in their native (left) and strand swapped configurations (right), showing strand G and hydrophobic residues (dark grey); (B) Swapping of strand A in Tencon (grey), showing the native conformation (left) and the 5 stranded β-sheet (right) and hydrophobic residues (dark grey); (C) The flexible N-terminus of Fibcon (grey), showing strand A and hydrophobic residues (dark grey).

FIGS. 11 A, B, C, D and E show a sequential and structural view of the determined important residues of groups A, B and C. (A) A sequence alignment of FN3con, Fibcon, FNfn10, Tencon, FNfn8 and TNfn3, highlighting group A residues (dark grey), group B residues (clear box), group C residues (grey) and respective AB, BC, CD, DE, EF and FG loops. (B) A cartoon represented figure of the FN3con crystal structure (PDB: 4U3H), highlighting the N/C termini and respective strand loops. (C) Positions of the tyrosine residues from group A residues. (D) Positions of L20, V22, V96 and V98 residues from group B. (E) Positions of the group C residues.

FIGS. 12 A, B and C show the design of grafted scaffold FN3con.DE0.4.1_graft using homology modeling. FIG. 12 A shows the DE0.4.1 homology model. FIG. 12 B shows the DE0.4.1 model alignment with FN3con and FIG. 12 C shows the loop boundaries used for rational grafting.

FIGS. 13 A and B show biophysical characterization of DE0.4.1 and FN3con.DE0.4.1_graft, namely protein folding and melting temperature.

FIGS. 14 A, B and C show the structure of DE0.4.1 complexed with lysozyme and that structure overlain with the FN3con scaffold to show regions of incompatibility.

FIG. 14 D shows a sequence alignment of FN3conDE0.4.1 and DE0.4.1 showing the binding interface identified by structure and the framework mutations introduced.

FIG. 15 shows flow cytometry titration of a FN3con library after 1 round of enrichment against biotinylated lysozyme.

### DETAILED DESCRIPTION

As used in this specification and the appended claims, the singular forms “a”, “an” and “the” include plural referents unless the context clearly dictates otherwise. The terms “a” (or “an”), as well as the terms “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this invention is related.

Units, prefixes, and symbols are denoted in their Systeme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or embodiments of the invention, which can be had by reference to the specification as a whole. Accordingly, the terms defined immediately below are more fully defined by reference to the specification in its entirety.

It is understood that wherever embodiments are described herein with the language “comprising,” otherwise analogous embodiments described in terms of “consisting of” and/or “consisting essentially of” are also provided.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter codes.

The present invention provides a fibronectin based scaffold with enhanced stability. Put another way the invention provides a polypeptide scaffold based on a fibronectin type III domain which has been modified to enhance the stability of the polypeptide.

The term “polypeptide” refers to any sequence of two or more amino acids, regardless of length, post-translation modification, or function. Polypeptides can include natural amino acids and non-natural amino acids. Polypeptides can also be modified in any of a variety of standard chemical ways (e.g., an amino acid can be modified with a protecting group; the carboxy-terminal amino acid can be made into a terminal amide group; the amino-terminal residue can be modified with groups to, e.g., enhance lipophilicity; or the polypeptide can be chemically glycosylated or otherwise modified to increase stability or in vivo half-life). Polypeptide modifications can include the attachment of another structure such as a cyclic compound or other molecule to the polypeptide and can also include polypeptides that contain one or more amino acids in an altered configuration (i.e., R or S; or, L or D).

As used herein, a “fibronectin based scaffold” or “FBS” protein or moiety refers to proteins or moieties that are based on a domain in fibronectin, the fibronectin type III (“Fn3”) repeat. Fn3 is a small (about 10 kDa) domain that has the structure of an immunoglobulin (Ig) fold (i.e., an Ig-like sandwich structure, consisting of seven n-strands and six loops). Fibronectin has 18 Fn3 repeats, and while the sequence homology between the repeats is low, they all share a high similarity in tertiary structure. Fn3 domains are also present in many proteins other than fibronectin, such as adhesion molecules, cell surface molecules, e.g., cytokine receptors, and carbohydrate binding domains. The term “fibronectin based scaffold” protein or moiety or “Fn3 protein” or “Fn3 domain” or “Fn3 domain protein” is intended to include proteins or domains based on Fn3 domains from these other (i.e., non-fibronectin) proteins.

Exemplary Fn3 domains include the 7th, 10th and 14th fibronectin type III domain, which are referred to herein as ⁷Fn3, ¹⁰Fn3 and ¹⁴Fn3, respectively and also as “reference scaffolds”. As used herein, a “Fn3 domain” or “Fn3 moiety” or “Fn3 domain protein” refers to wild-type Fn3 and biologically active variants thereof, e.g., biologically active variants that may specifically bind to a target, such as EGFR, IL23 and IGFIR. For example, ¹⁰Fn3 molecules binding to specific targets may be selected from ¹⁰Fn3 libraries using the PROfusion technique described in WO02/32925. Wild type Fn3 domains upon which the polypeptide scaffold can be based are exemplified by the amino acid sequences set forth in SEQ ID NOs: 3-8.

### Examples of FN3 Domain Polypeptide Scaffold Sequences

Wild type (WT):
WT human ¹⁰Fn3 Domain:
(SEQ ID NO: 3)
VSDVPRDLEVVAATPTSLLISWDAPAVTVRYYRITYGETGGNSPVQEF
TVPGSKSTATISGLKPGVDYTITVYAVTGRGDSPASSKPISINYRTEID
KPSQ
WT human ⁷Fn3 Domain:
(SEQ ID NO: 4)
PLSPPTNLHLEANPDTGVLTVSWERSTTPDITGYRITTTPTNGQQGNSL
EEVVHADQSSCTFDNLSPGLEYNVSVYTVKDDKESVPISDTIIP
WT human ¹⁴Fn3 Domain:
(SEQ ID NO: 5)
NVSPPRRARVTDATETTITISWRTKTETITGFQVDAVPANGQTPIQRTIK
PDVRSYTITGLQPGTDYKIYLYTLNDNARSSPVVIDAST
TNfn3
(SEQ IN NO: 6)
RLDAPSQIEVKDVTDTTALITWFKPLAEIDGIELTYGIKDVPGDRTTIDL
TEDENQYSIGNLKPDTEYEVSLISRRGDMSSNPAKETFTT
FNfn8
(SEQ ID NO: 7)
PPTDLRFTNIGPDTMRVTWAPPPSIDLTNFLVRYSPVKNEEDVAELSISP
SDNAVVLTNLLPGTEYVVSVSSVYEQHESTPLRGRQKT
FNfn10
(SEQ ID NO: 8)
VSDVPRDLEVVAATPTSLLISWDAPAVTVRYYRITYGETGGNSPVQEFTV
PGSKSTATISGLKPGVDYTITVYAVTGRGDSPASSKPISINYRT
Engineered
Tencon
(SEQ ID NO: 9)
MLPAPKNLVVSEVTEDSLRLSWTAPDAAFDSFMIQYQESEKVGEAINLTV
PGSERSYDLTGLKPGTEYTVSIYGVKGGHRSNPLSAEFTTGG
Fibcon
(SEQ ID NO: 10)
MLDAPTDLQVTNVTDTSITVSWTPPSATITGYRITYTPSNGPGEPKELTV
PPSSTSVTITGLTPGVEYVVSVYALKDNQESPPLVGTQTTGG

The phrase “comprising an amino acid sequence based on” a specific or first sequence is intended to include amino acid sequences that are derived from the specific or first amino acid sequence, e.g., by amino acid substitutions, additions or deletions. For example, a protein comprising an amino acid sequence based on an amino acid sequence selected from SEQ ID NOs: 3-5 refers to a protein comprising an amino acid sequence that is derived from any of SEQ ID NOs: 3-5, including, e.g., a protein comprising an amino acid sequence that differs from one or more of SEQ ID NOs: 3-5 in one or more loop or non-loop sequences, such as to obtain binding to a desired target.

### Stability Enhancing Modifications

The inventors have determined that various modifications to the Fn3 domain provide a polypeptide scaffold that is more stable than the wild type Fn3 domain. Comparative studies provided herein show that certain polypeptide scaffolds exemplified herein are more stable than any other Fn3 based polypeptide scaffolds seen to date.

In one embodiment the stable polypeptide scaffold of the invention comprises a Fn3 domain that comprise at least, at most or about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acid variations, i.e., substitutions, additions or deletions, relative to a Fn3 domain comprising an amino acid sequence selected from SEQ ID NOs: 7-10.

The polypeptide scaffold may further comprise or comprise at most, 1, 2, 3, 4, 5, 6, 7, 8, 9, 10, 15, 20, 25, 30 or 40 amino acid changes relative to the polypeptide scaffold of SEQ ID NO: 1 or SEQ ID NO: 2. In a preferred embodiment said amino acid changes are present in one or more loop regions. In a preferred embodiment changes not in loop regions comprise conservative substitutions.

In one embodiment the polypeptide scaffold comprises one or more further amino acid changes relative to the polypeptide scaffold sequence of SEQ ID NO: 1 or SEQ ID NO: 2 which change(s) protect the hydrophobic core via mediation of interactions with water.

In one embodiment the polypeptide scaffold comprises one or more further amino acid changes relative to the polypeptide scaffold sequence of SEQ ID NO: 1 or SEQ ID NO: 2 which change(s) provide favourable interactions amongst the N and C terminal strands of the molecule at elevated temperatures.

In one embodiment the polypeptide scaffold comprises one or more further amino acid changes relative to the polypeptide scaffold sequence of SEQ ID NO: 1 or SEQ ID NO: 2 which change(s) provide a complementary salt bridge network on the surface of β-sheet 2 which restricts unfolding of the molecule at high temperatures.

Polypeptide scaffolds possess many of the properties of antibodies in relation to their fold that mimics the variable region of an antibody. This orientation enables the FN3 loops to be exposed similar to antibody complementarity determining regions (CDRs). They should be able to bind to cellular targets and the loops can be altered, e.g., affinity matured, to improve certain binding or related properties.

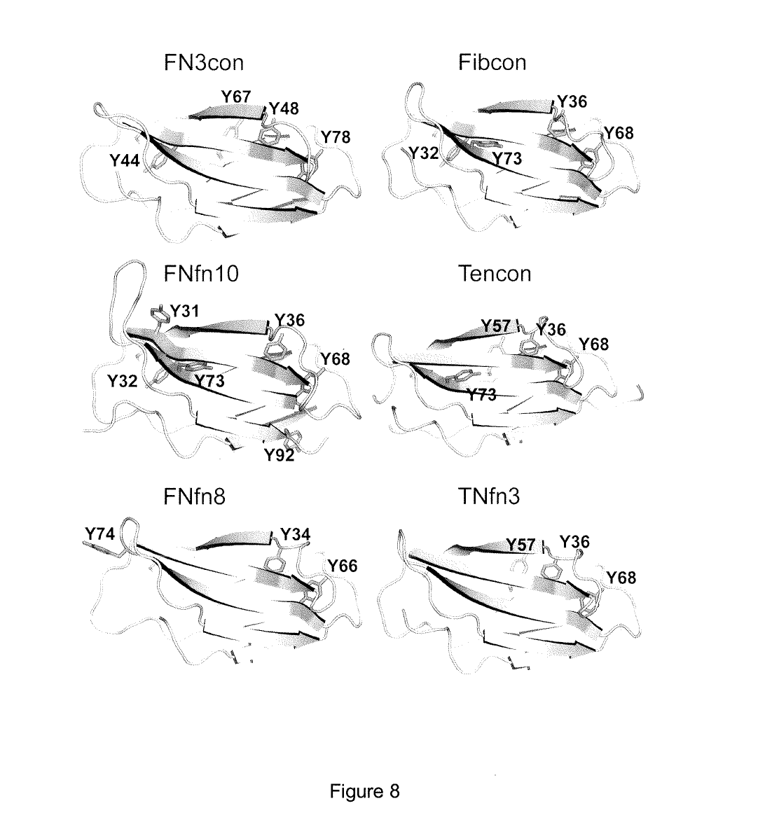

Three of the six loops of the polypeptide scaffold of the invention correspond topologically to the binding domains of an antibody positioned at the loops of the variable domain known to be hypervariable in nature (the hypervariable domains loops (HVL), at positions as defined by Kabat as the residues of the complementarity determining regions (CDRs), i.e., antigen-binding regions, of an antibody, while the remaining three loops are surface exposed in a manner similar to antibody CDRs. These loops span or are positioned at or about residues 26-29, 36-42, 50-54, 61-65, 70-76, and 85-93 of SEQ ID NO:2 as shown in FIGS. 8 and 11. Preferably, one or more of the loop regions at or about residues 36-42, 61-65, and 85-93 are altered for binding specificity and affinity. One or more of these loop regions are randomized with other loop regions and/or other strands maintaining their sequence as backbone portions to populate a library and potent binders can be selected from the library having high affinity for a particular protein target. One or more of the loop regions can interact with a target protein similar to an antibody CDR interaction with the protein.

In one embodiment one or more of the loops regions at or about residues 36-42, 61-65, and 85-93 of SEQ ID NO:2 are altered to include loops from previously established FN3 domains that can bind to lysozyme at 1 μM and VEGFR-2 (CT-22).

In one embodiment one or more of the loops regions at or about residues 36-42, 61-65, and 85-93 of SEQ ID NO:2 are altered to include loops with affinity to targets that are typically difficult to target (low specificity, and or low affinity). These could be targets that are infectious diseases, chemical warfare agents, toxins (peptide and small molecule), markers present in bodily fluids, for example blood, serum, urine, tears, such as heart disease related markers in blood and cancer related markers in blood. This embodiment provides diagnostic reagents that can be used to detect and almost anything that antibodies can detect but which are capable of storage and use in environments where cold chain storage is not available.

In one embodiment one or more of the loops regions at or about residues 36-42, 61-65, and 85-93 of SEQ ID NO:2 are altered to include loops which make the scaffold suitable as a reagent for protein detection, immunoprecipitation/purification, agonists/antagonists and co-crystallants. Again the scaffold is adapted to act in the same way as an antibody reagent but provides the advantage of potentially cheaper development cost for an FN3 domain, cheaper production costs, greater stability than antibodies, so shelf lives are longer and with higher binding affinities (potentially in the fM range) and higher specificities with negative selection in the generation process.

In one embodiment one or more of the loops regions at or about residues 36-42, 61-65, and 85-93 of SEQ ID NO:2 are altered to include loops directed to G protein coupled receptors for targeting extracellular and intracellular tails, targeting their active sites with functional selections and usage as co-crystallants in lipid cubic phase.

In one embodiment one or more of the loops regions at or about residues 36-42, 61-65, and 85-93 of SEQ ID NO:2 are altered to include loops for a therapeutic agent. The loops may target everything from topical receptors (skin diseases, mouth diseases, etc) through to very high impact targets, PD1's (anti-cancer), Gp41 (anti-HIV), infectious diseases and anything an antibody targets. It may also be possible to attach various flavours of the Fc domains to the scaffold to induce immune responses/cell death.

In certain embodiments the beta strands of the polypeptide scaffold exhibit at least 50%, at least 55%, at least 60%, at least 65%, at least 70%, at least 75%, at least 80%, at least 85%, at least 90%, at least 95%, at least 99% or more sequence identity to the primary sequences of the beta strands of SEQ ID NOs: 1 or 2 or to the primary sequences of the beta strands of any of the Fn3 domains or to the beta strands of a protein domain recognized to contain the Interpro IPR008957 fibronectin type III domain signature as determined using the InterProScan program, or recognized to contain the Pfam PF00041 fibronectin type III domain signature as determined using Pfam scan, HMMER, or any other program capable of comparing a protein sequence to a Hidden Markov model.

The term “sequence homology” in relation to protein sequences refers to the similarity between two or more protein sequences, i.e., the percentage of amino acid residues that are either identical or conservative amino acid substitutions.

The terms “Percent (%) sequence similarity” and “Percent (%) homology” as used herein are considered equivalent and are defined as the percentage of amino acid residues in a candidate sequence that are identical with or conservative substitutions of the amino acid residues in a selected sequence, after aligning the amino acid sequences and introducing gaps in the candidate and/or selected sequences, if necessary, to achieve the maximum percent sequence similarity.

“Percent (%) identity” is defined herein as the percentage of amino acid residues in a candidate sequence that are identical with the amino acid residues in a selected sequence, after aligning the sequences and introducing gaps in the candidate and/or selected sequence, if necessary, to achieve the maximum percent sequence identity, and not considering any conservative amino acid substitutions as part of the sequence identity.

The term “conservative substitution” as used herein denotes the replacement of an amino acid residue by another, biologically similar residue. Examples of conservative substitutions include the substitution of one hydrophobic amino acid residue such as isoleucine, valine, leucine, alanine, cysteine, glycine, phenylalanine, proline, tryptophan, tyrosine, norleucine, or methionine for another, or the substitution of one polar residue for another, such as the substitution of arginine for lysine and vice versa, of glutamic acid for aspartic acid, and vice versa, glutamine for asparagine, and vice versa, and the like. Neutral hydrophilic amino acids which can be substituted for one another include asparagine, glutamine, serine and threonine. The term “conservative substitution” also includes the use of a substituted amino acid in place of an unsubstituted parent amino acid provided that the biologic activity of the peptide is maintained. Biological similarity between amino acid residues refers to similarities between properties such as, but not limited to, hydrophobicity, mutation frequency, charge, side chain length, size chain volume, pKa, polarity, aromaticity, solubility, surface area, peptide bond geometry, secondary structure propensity, average solvent accessibility, etc.

Alignment for purposes of determining percent homology (i.e. sequence similarity) or percent identity can be achieved in various ways that are within the skill in the art, for instance, using publicly or proprietary algorithms. For instance, sequence similarity can be determined using pairwise alignment methods, e.g., BLAST, BLAST-2, ALIGN, or ALIGN-2 or multiple sequence alignment methods such as Megalign (DNASTAR), ClustalW or T-Coffee software. Those skilled in the art can determine appropriate scoring functions, e.g., gap penalties or scoring matrices for measuring alignment, including any algorithms needed to achieve optimal alignment quality over the full-length of the sequences being compared. Furthermore, those skilled in the art would appreciate that methods to identify proteins with a certain fold, e.g., the Fn3 fold, and to align the amino acid sequences of such proteins, include sequence-sequence methods, sequence-profile methods, and profile-profile methods. In addition, sequence alignment can be achieved using structural alignment methods (e.g., methods using secondary or tertiary structure information to align two or more sequences), or hybrid methods combining sequence, structural, and phylogenetic information to identify and optimally align candidate protein sequences.

FIG. 11 shows a sequence alignment between the polypeptide scaffold of the first aspect and Fn3 based polypeptides known in the art.

The term “stability” as used herein refers to the ability of a molecule to maintain a folded state under physiological conditions such that it retains at least one of its normal functional activities, for example, binding to a target molecule like a cytokine or serum protein. Measurement of protein stability and protein liability can be viewed as the same or different aspects of protein integrity. Proteins are sensitive or “labile” to denaturation caused by heat, by ultraviolet or ionizing radiation, changes in the ambient osmolarity and pH if in liquid solution, mechanical shear force imposed by small pore-size filtration, ultraviolet radiation, ionizing radiation, such, as by gamma irradiation, chemical or heat dehydration, or any other action or force that may cause protein structure disruption.

The stability of a molecule can be determined using standard methods. For example, the stability of a molecule can be determined by measuring the thermal melt (“Tm”) temperature. The Tm is the temperature in degrees Celsius (° C.) at which half of the molecules become unfolded. Typically, the higher the Tm, the more stable the molecule. In addition to heat, the chemical environment also changes the ability of the protein to maintain a particular three dimensional structure.

Chemical denaturation can likewise be measured by a variety of methods. A chemical denaturant is an agent known to disrupt non-covalent interactions and covalent bonds within a protein, including hydrogen bonds, electro-static bonds, Vander Waals forces, hydrophobic interactions, or disulfide bonds. Chemical denaturants include guanidinium hydrochloride, guanadinium thiocyanate, urea, acetone, organic solvents (DMF, benzene, acetonitrile), salts (ammonium sulfate lithium bromide, lithium chloride, sodium bromide, calcium chloride, sodium chloride); reducing agents (e.g. dithiothreitol, beta-mercaptoethanol, dinitrothiobenzene, and hydrides, such as sodium borohydride), non-ionic and ionic detergents, acids (e.g. hydrochloric acid (HCl), acetic acid (CH3COOH), halogenated acetic acids), hydrophobic molecules (e.g. phosopholipids), and targeted denaturants. Quantitation of the extent of denaturation can rely on loss of a functional property such as ability to bind a target molecule, or by physiochemical properties such tendency to aggregation, exposure of formerly solvent inaccessible residues, or disruption or formation of disulfide bonds.

“Denaturing” or “denaturation” of a protein is the process where some or all of the three-dimensional conformation imparting the functional properties of the protein has been lost with an attendant loss of activity and/or solubility. Forces disrupted during denaturation include intramolecular bonds, including but not limited to electrostatic, hydrophobic, Van der Waals forces, hydrogen bonds, and disulfides. Protein denaturation can be caused by forces applied to the protein or a solution comprising the protein such as mechanical force (for example, compressive or shear-force), thermal, osmotic stress, change in pH, electrical or magnetic fields, ionizing radiation, ultraviolet radiation and dehydration, and by chemical denaturants. In some embodiments the increased protein stability of the polypeptide scaffolds is measured by differential scanning calorimetry (DSC), circular dichroism (CD), polyacrylamide gel electrophoresis (PAGE), protease resistance, isothermal calorimetry (ITC), nuclear magnetic resonance (NMR), urea denaturation, or guanidine denaturation.

In one embodiment the stable polypeptide scaffolds of the invention exhibit an increase in stability of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% or more compared to the reference scaffold, as measured by thermal tolerance, resistance to chaotropic denaturation, protease treatment or another stability parameter well-known in the art.

The stability of a protein may be measured by the level of fluorescence exhibited by the protein under varying conditions. There is a positive correlation between the relative unfoldedness of a protein and a change in the internal fluorescence the protein exhibits under stress. Suitable protein stability assays to measure thermal unfolding characteristics include Differential Scarming calorimetry (DSC) and Circular Dichroism (CD). When the protein demonstrates a sizable shift in parameters measured by DSC or CD, it correlates to an unfolded structure. The temperature at which this shift is made is termed the melting temperature or (Tm).

In one embodiment, the stable polypeptide scaffolds of the invention exhibit an increased melting temperature (Tm) of at least 1° C., at least 2° C., at least 3° C., at least 4° C., at least 5° C., at least 10° C., at least 15° C., at least 20° C., at least 25° C., at least 30° C., at least 35° C., at least 45° C., at least 50° C., at least 55° C., at least 60° C., at least 65° C., at least 70° C., at least 71° C., at least 72° C., at least 73° C., at least 74° C., at least 75° C., at least 76° C., at least 77° C., at least 78° C., at least 79° C., at least 80° C., at least 81° C., at least 82° C., at least 83° C., at least 84° C., at least 85° C., at least 85° C., at least 86° C., at least 87° C., at least 88° C., at least 89° C., at least 90° C., at least 91° C., at least 92° C., at least 93° C., at least 94° C., at least 94° C., at least, at least 95° C., at least 96° C., at least 97° C., at least 98° C., at least 100° C., at least 105° C., at least 110° C., or at least 120° C. as compared to reference scaffold under similar conditions.

In another embodiment, the stable polypeptide scaffolds of the invention exhibit an increased melting temperature (Tm) of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% or more as compared to the reference scaffold under similar conditions.

Another assay for protein stability involves exposing a protein to a chaotropic agent, such as urea or guanidine (for example, guanidine-HCl or guanidine isothiocynate) which acts to destabilize interactions within the protein. Upon exposing the protein to increasing levels of urea or guanidine, the relative intrinsic fluorescence is measured to assess a value in which 50% of the protein molecules are unfolded. This value is termed the Cm value and represents a benchmark value for protein stability. The higher the Cm value, the more stable the protein. In one embodiment, the stable polypeptide scaffolds of the invention exhibit an increased Cm at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% or more as compared to the reference scaffold as measured in a urea denaturation experiment under similar conditions. In another embodiment, the stable polypeptide scaffolds of the invention exhibit an increased Cm at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% or more as compared to the reference scaffold as measured in a guanidinium-HCl denaturation experiment under similar conditions.

Another assay used to assay protein stability is a protease resistance assay. In this assay, a relative level of protein stability is correlated with the resistance to protease degradation over time. The more resistant to protease treatment, the more stable the protein is. In one embodiment, the stable polypeptide scaffolds of the invention exhibit increased stability by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% or more as compared to the reference scaffold under similar conditions.

The protein scaffolds of the present invention are more stable than reference scaffolds under conditions of high osmotic strength. In one embodiment the protein scaffolds of the present invention are also more soluble at high concentrations than the reference scaffolds.

The polypeptide scaffolds of the present invention may incorporate other subunits, e.g., via covalent interaction. All or a portion of an antibody constant region may be attached to the scaffold to impart antibody-like properties especially those properties associated with the Fc region, e.g., complement activity (ADCC), half-life, etc. For example, effector function can be provided and/or controlled, e.g., by modifying C1q binding and/or FcγR binding and thereby changing CDC activity and/or ADCC activity.

“Effector functions” are responsible for activating or diminishing a biological activity (e.g., in a subject). Examples of effector functions include, but are not limited to: C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions may require the Fc region to be combined with a binding domain (e.g., protein scaffold loops) and can be assessed using various assays (e.g., Fc binding assays, ADCC assays, CDC assays, etc.).

Additional moieties may be appended or associated with the polypeptide scaffold, such as a toxin conjugate, albumin or albumin binders, polyethylene glycol (PEG) molecules may be attached to the scaffold molecule for desired properties. These moieties may be in-line fusions with the scaffold coding sequence and may be generated by standard techniques, for example, by expression of the fusion protein from a recombinant fusion encoding vector constructed using publicly available coding nucleotide sequences. Alternatively, chemical methods may be used to attach the moieties to a recombinantly produced polypeptide scaffold.

The polypeptide scaffolds of the present invention can be used as monospecific in monomeric form or as bi- or multi-specific (for different protein targets or epitopes on the same protein target) in multimer form. The attachments between each scaffold unit may be covalent or non-covalent. For example, a dimeric bispecific scaffold has one subunit with specificity for a first target protein or epitope and a second subunit with specificity for a second target protein or epitope. Scaffold subunits can be joined in a variety of conformations that can increase the valency and thus the avidity of antigen binding.

### Generation and Production of Scaffold Protein

The polypeptide scaffolds of the invention may be produced by a cell line, a mixed cell line, an immortalized cell or clonal population of immortalized cells, as well known in the art.

Amino acids of the polypeptide scaffold of the present invention may be altered, added and/or deleted to reduce immunogenicity or reduce, enhance or modify binding, affinity, on-rate, off-rate, avidity, specificity, half-life, stability, solubility or any other suitable characteristic, as known in the art.

Bioactive polypeptide scaffolds can be engineered with retention of high affinity for the antigen and other favorable biological properties. To achieve this goal, the polypeptide scaffolds can be optionally prepared by a process of analysis of the parental sequences and various conceptual engineered products using three-dimensional models of the parental and engineered sequences. Three-dimensional models are commonly available and are familiar to those skilled in the art. Computer programs are available which illustrate and display probable three-dimensional conformational structures of selected candidate sequences and can measure possible immunogenicity (e.g., Immunofilter program of Xencor, Inc. of Monrovia, Calif.). Inspection of these displays permits analysis of the likely role of the residues in the functioning of the candidate sequence, i.e., the analysis of residues that influence the ability of the candidate scaffold protein to bind its antigen. In this way, residues can be selected and combined from the parent and reference sequences so that the desired characteristic, such as affinity for the target antigen(s), is achieved. Alternatively, or in addition to, the above procedures, other suitable methods of engineering can be used.

### Screening

Screening polypeptide scaffolds of the invention or libraries comprising such polypeptide scaffolds with variegated residues or domains for specific binding to similar proteins or fragments can be conveniently achieved using nucleotide (DNA or RNA display) or peptide display libraries, for example, in vitro display. This method involves the screening of large collections of peptides for individual members having the desired function or structure. The displayed peptide with or without nucleotide sequences can be from 3 to 5000 or more nucleotides or amino acids in length, frequently from 5-100 amino acids long, and often from about 8 to 25 amino acids long. In addition to direct chemical synthetic methods for generating peptide libraries, several recombinant DNA methods have been described. One type involves the display of a peptide sequence on the surface of a bacteriophage or cell. Each bacteriophage or cell contains the nucleotide sequence encoding the particular displayed peptide sequence.

The polypeptide scaffolds of the invention can bind human or other mammalian proteins with a wide range of affinities (K_{D}). In a preferred embodiment, at least one polypeptide scaffold of the present invention can optionally bind to a target protein with high affinity, for example, with a K_{D} equal to or less than about 10⁻⁷M, such as but not limited to, 0.1-9.9 (or any range or value therein)×10⁻⁸, 10⁻⁹, 10⁻¹⁰, 10⁻¹¹, 10⁻¹², 10⁻¹³, 10⁻¹⁴, 10⁻¹⁵ or any range or value therein, as determined by surface plasmon resonance or the Kinexa method, as practiced by those of skill in the art.

The affinity or avidity of a polypeptide scaffold for an antigen can be determined experimentally using any suitable method. The measured affinity of a particular scaffold-antigen interaction can vary if measured under different conditions (e.g., osmolarity, pH). Thus, measurements of affinity and other antigen-binding parameters (e.g., K_{D}, Kₒₙ, K_{off}) are preferably made with standardized solutions of scaffold and antigen, and a standardized buffer, such as the buffer described herein.

Competitive assays can be performed with the polypeptide scaffold of the present invention in order to determine what proteins, antibodies, and other antagonists compete for binding to a target protein with the scaffold of the present invention and/or share the epitope region. These assays as readily known to those of ordinary skill in the art evaluate competition between antagonists or ligands for a limited number of binding sites on a protein. The protein and/or antibody is immobilized, isolated, or captured before or after the competition and the sample bound to the target protein is separated from the unbound sample, for example, by decanting (where the protein/antibody was preinsolubilized) or by centrifuging (where the protein/antibody was precipitated after the competitive reaction). Also, the competitive binding may be determined by whether function is altered by the binding or lack of binding of the protein scaffold to the target protein, e.g., whether the protein scaffold molecule inhibits or potentiates the enzymatic activity of, for example, a label. ELISA and other functional assays may be used, as well known in the art.

### Nucleic Acid Molecules

Nucleic acid molecules encoding the polypeptide scaffolds or polypeptides of the invention, can be in the form of RNA, such as mRNA, hnRNA, tRNA or any other form, or in the form of DNA, including, but not limited to, cDNA and genomic DNA obtained by cloning or produced synthetically, or any combinations thereof. The DNA can be triple-stranded, double-stranded or single-stranded, or any combination thereof. Any portion of at least one strand of the DNA or RNA can be the coding strand, also known as the sense strand, or it can be the non-coding strand, also referred to as the anti-sense strand.

Isolated nucleic acid molecules of the present invention can include nucleic acid molecules comprising an open reading frame (ORF), optionally, with one or more introns, e.g., but not limited to, at least one specified portion of at least one polypeptide scaffold; nucleic acid molecules comprising the coding sequence for a polypeptide scaffold or loop region that binds to the target protein; and nucleic acid molecules which comprise a nucleotide sequence substantially different from those described above but which, due to the degeneracy of the genetic code, still encode the polypeptide scaffold as described herein and/or as known in the art.

Thus, it would be routine for one skilled in the art to generate such degenerate nucleic acid variants that code for specific protein scaffolds of the present invention.

As indicated herein, nucleic acid molecules of the present invention may also comprise additional sequences, such as the coding sequence of at least one signal leader or fusion peptide, with or without the aforementioned additional coding sequences, such as at least one intron, together with additional, non-coding sequences, including but not limited to, non-coding 5′ and 3′ sequences, such as the transcribed, non-translated sequences that play a role in transcription, mRNA processing, including splicing and polyadenylation signals (for example, ribosome binding and stability of mRNA); an additional coding sequence that codes for additional amino acids, such as those that provide additional functionalities. Thus, the sequence encoding a protein scaffold can be fused to a marker sequence, such as a sequence encoding a peptide that facilitates purification of the fused protein scaffold comprising a protein scaffold fragment or portion.

The invention also provides for nucleic acids encoding the compositions of the invention as isolated polynucleotides or as portions of expression vectors including vectors compatible with prokaryotic, eukaryotic or filamentous phage expression, secretion and/or display of the compositions or directed mutagens thereof.

The isolated nucleic acids of the present invention can be made using (a) recombinant methods, (b) synthetic techniques, (c) purification techniques, and/or (d) combinations thereof, as well-known in the art.

The isolated nucleic acid molecules of the present invention encode a functional portion of the polypeptide scaffold described herein. The polynucleotides of this invention embrace nucleic acid sequences that can be employed for selective hybridization to a polynucleotide encoding a protein scaffold of the present invention. The present invention provides isolated nucleic acids that hybridize under selective hybridization conditions to a polynucleotide disclosed herein. Thus, the polynucleotides of this embodiment can be used for isolating, detecting, and/or quantifying nucleic acids comprising such polynucleotides. For example, polynucleotides of the present invention can be used to identify, isolate, or amplify partial or full-length clones in a deposited library. In some embodiments, the polynucleotides are genomic or cDNA sequences isolated, or otherwise complementary to, a cDNA from a human or mammalian nucleic acid library.

The nucleic acids can conveniently comprise sequences in addition to a polynucleotide of the present invention. For example, a multi-cloning site comprising one or more endonuclease restriction sites can be inserted into the nucleic acid to aid in isolation of the polynucleotide. Also, translatable sequences can be inserted to aid in the isolation of the translated polynucleotide of the present invention.

A cDNA or genomic library can be screened using a probe based upon the sequence of a polynucleotide of the present invention, such as those disclosed herein. Probes can be used to hybridize with genomic DNA or cDNA sequences to isolate homologous genes in the same or different organisms. Those of skill in the art will appreciate that various degrees of stringency of hybridization can be employed in the assay; and either the hybridization or the wash medium can be stringent. As the conditions for hybridization become more stringent, there must be a greater degree of complementarity between the probe and the target for duplex formation to occur. The degree of stringency can be controlled by one or more of temperature, ionic strength, pH and the presence of a partially denaturing solvent, such as formamide. For example, the stringency of hybridization is conveniently varied by changing the polarity of the reactant solution through, for example, manipulation of the concentration of formamide within the range of 0% to 50%. The degree of complementarity (sequence identity) required for detectable binding will vary in accordance with the stringency of the hybridization medium and/or wash medium. The degree of complementarity will optimally be 100%, or 70-100%, or any range or value therein. However, it should be understood that minor sequence variations in the probes and primers can be compensated for by reducing the stringency of the hybridization and/or wash medium.

In one aspect of the invention, the polynucleotides are constructed using techniques for incorporation of randomized codons in order to variegate the resulting polypeptide at one or more specific residues or to add residues at specific locations within the sequence. Various strategies may be used to create libraries of altered polypeptide sequences including random, semi-rational and rational methods. Rational and semi-rational methods have the advantage over the random strategies in that one has more control over the consequences of changes introduced into the coding sequence. In addition, by focusing the variation in certain regions of the gene, the universe of all possible amino acid variants can be explored in chosen positions. A library built on the common NNK or NNS diversification scheme introduce a possible 32 different codons in every position and all 20 amino acids. Such a library theoretically grows by 32n for every n number of residues. In practical terms, however, phage display is limited to sampling libraries of 10⁹ to 10¹⁰ variants implying that only 6-7 residues can be targeted for variegation if full sequence coverage is to be achieved in the library. Thus, semi-rational or “focused” methods to generate libraries of scaffold variants by identifying key positions to be variegated and choosing the diversification regime according can be applied. A “codon set” refers to a set of different nucleotide triplet sequences used to encode desired variant amino acids. A standard form of codon designation is that of the IUB code, which is known in the art and described herein. A “non-random codon set” refers to a codon set that encodes select amino acids. Synthesis of oligonucleotides with selected nucleotide “degeneracy” at certain positions is well known in that art, for example the TRIM approach (Knappek et al.; J. Mol. Biol. (1999), 296:57-86); Garrard & Henner, Gene (1993), 128:103). Such sets of nucleotides having certain codon sets can be synthesized using commercially available nucleotide or nucleoside reagents and apparatus.

Standard cloning techniques are used to clone the libraries into a vector for expression. The library may be expressed using known system, for example expressing the library as fusion proteins. The fusion proteins can be displayed on the surface of any suitable phage. Methods for displaying fusion polypeptides comprising antibody fragments on the surface of a bacteriophage are well known. Libraries for de novo polypeptide isolation can be displayed on pIX. The libraries can also be translated in vitro, for example using ribosome display, mRNA display, CIS-display or other cell-free systems.

Libraries with diversified regions can be generated using vectors comprising the polynucleotide encoding the Fn3con sequence (SEQ ID NO: 2) or a predetermined mutant thereof. The template construct may have a promoter and signal sequences for the polypeptide chain. To make scaffold libraries, mutagenesis reactions using oligonucleotides that coded for loop regions (A:B, B:C, C:D, D:E, E:F, and F:G) of the scaffold are used. To ensure the incorporation of all chosen positions into the randomization scheme, a stop codon (such as TAA) can be incorporated in each region desired to be intended to be diversified. Only clones where the stop codons have been replaced will occur.

In one embodiment the library is a yeast surface display library comprising a scaffold into which codons for the BC, DE and/or FG loops or a portion thereof are replaced with degenerate NNS codons.

In one embodiment the library is a FN3con.NNS library as depicted in SEQ ID NO: 11, in which X depicts a degenerate NNS codon.

(SEQ ID NO: 11)
MPSPPGNLRVTDVTSTSVTLSWEXXXXXXXGYRVEYREAGGEWKEVTVPX
XXXXSYTVTGLKPGTEYEERVRAXXXXXXXXPSSVSVTT.

### Modified Polypeptide Scaffolds

Modified polypeptide scaffolds and fragments of the invention can comprise one or more moieties that are covalently bonded, directly or indirectly, to another protein.

In the case of the addition of peptide residues, or the creation of an in-line fusion protein, the addition of such residues may be through recombinant techniques from a polynucleotide sequence as described herein. In the case of an appended, attached or conjugated peptide, protein, organic chemical, inorganic chemical or atom, or any combination thereof, the additional moiety that is bonded to a polypeptide scaffold or fragment of the invention is typically via other than a peptide bond. The modified polypeptide scaffolds of the invention can be produced by reacting a polypeptide scaffold or fragment with a modifying agent. For example, the organic moieties can be bonded to the polypeptide scaffold in a non-site specific manner by employing an amine-reactive modifying agent, for example, an NHS ester of PEG. Modified polypeptide scaffolds and fragments comprising an organic moiety that is bonded to specific sites of a polypeptide scaffold of the present invention can be prepared using suitable methods, such as reverse proteolysis.

Where a polymer or chain is attached to the polypeptide scaffold, the polymer or chain can independently be a hydrophilic polymeric group, a fatty acid group or a fatty acid ester group. As used herein, the term “fatty acid” encompasses mono-carboxylic acids and di-carboxylic acids. A “hydrophilic polymeric group,” as the term is used herein, refers to an organic polymer that is more soluble in water than in octane. For example, polylysine is more soluble in water than in octane. Thus, a polypeptide scaffold modified by the covalent attachment of polylysine is encompassed by the invention. Hydrophilic polymers suitable for modifying polypeptide scaffolds of the invention can be linear or branched and include, for example, polyalkane glycols (e.g., PEG, monomethoxy-polyethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. Preferably, the hydrophilic polymer that modifies the polypeptide scaffold of the invention has a molecular weight of about 800 to about 150,000 Daltons as a separate molecular entity. For example, PEG₅₀₀₀ and PEG_{20,000}, wherein the subscript is the average molecular weight of the polymer in Daltons, can be used. The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N,N-carbonyl diimidazole) on a fatty acid or fatty acid ester can be coupled to a hydroxyl group on a polymer.

Fatty acids and fatty acid esters suitable for modifying polypeptide scaffolds of the invention can be saturated or can contain one or more units of unsaturation. Fatty acids that are suitable for modifying polypeptide scaffolds of the invention include, for example, n-dodecanoate (C₁₂, laurate), n-tetradecanoate (C₁₄, myristate), n-octadecanoate (C₁₈, stearate), n-eicosanoate (C₂₀, arachidate), n-docosanoate (C₂₂, behenate), n-triacontanoate (C₃₀), n-tetracontanoate (C₄₀), cis-Δ9-octadecanoate (C₁₈, oleate), all cis-Δ5,8,11,14-eicosatetraenoate (C20, arachidonate), octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably, one to about six, carbon atoms.

### Host Cell Selection or Host Cell Engineering

As described herein, the host cell chosen for expression of the polypeptide scaffold is an important contributor to the final composition, including, without limitation, the variation in composition of the oligosaccharide moieties decorating the protein, if desirable, for example in the immunoglobulin CH2 domain when present. Thus, one aspect of the invention involves the selection of appropriate host cells for use and/or development of a production cell expressing the desired therapeutic protein.

Further, the host cell may be of mammalian origin or may be selected from COS-1, COS-7, HEK293, BHK21, CHO, BSC-1, Hep G2, 653, SP2/0, 293, HeLa, myeloma, lymphoma, yeast, insect or plant cells, or any derivative, immortalized or transformed cell thereof. Alternatively, the host cell may be selected from a species or organism incapable of glycosylating polypeptides, e.g. a prokaryotic cell or organism, such as and of the natural or engineered E. coli spp, Klebsiella spp., or Pseudomonas spp.

### Uses of Scaffold-Based Molecules

The polypeptide scaffolds described herein may be used to diagnose, monitor, modulate, treat, alleviate, help prevent the incidence of, or reduce the symptoms of disease or specific pathologies in cells, tissues, organs, fluid, or, generally, a host. A polypeptide scaffold engineered for a specific purpose may be used to treat an immune-mediated or immune-deficiency disease, a metabolic disease, a cardiovascular disorder or disease; a malignant disease; neurologic disorder or disease; an infection such as a bacterial, viral or parasitic infection; or other known or specified related condition including swelling, pain, and tissue necrosis or fibrosis.

Such a method can comprise administering an effective amount of a composition or a pharmaceutical composition comprising at least one polypeptide scaffold to a cell, tissue, organ, animal or human patient in need of such modulation, treatment, alleviation, prevention, or reduction in symptoms, effects or mechanisms. The effective amount can comprise an amount of about 0.001 to 500 mg/kg per single (e.g., bolus), multiple or continuous administration, or to achieve a serum concentration of 0.01-5000 μg/ml serum concentration per single, multiple, or continuous administration, or any effective range or value therein, as done and determined using known methods, as described herein or known in the relevant arts.

Compositions Comprising Stable Polypeptide Scaffolds

For therapeutic use, the polypeptide scaffolds may be formulated of an appropriate mode of administration including but not limited to parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal means.

At least one polypeptide scaffold can be prepared for use in the form of tablets or capsules; powders, nasal drops or aerosols; a gel, ointment, lotion, suspension or incorporated into a therapeutic bandage or “patch” delivery system as known in the art.

The invention provides stable polypeptide scaffold formulations, which is preferably an aqueous phosphate buffered saline or mixed salt solution, as well as preserved solutions and formulations as well as multi-use preserved formulations suitable for pharmaceutical or veterinary use, comprising at least one polypeptide scaffold in a pharmaceutically acceptable formulation. Suitable vehicles and their formulation, inclusive of other human proteins, e.g., human serum albumin, are described in the art. The compositions may be used with, or incorporate within a single formulation, other actives known to be beneficial for treatment of the indicated disorder, condition, or disease or may be a tested by preparing combinations of polypeptide scaffolds with novel compositions and actives.

### EXAMPLES

The invention described herein will be more readily understood by reference to the following examples which are included merely for purposes of illustration of certain aspects and embodiments of the present disclosure, and are not intended to limit the invention in any way.

### Example 1

The amino acid sequence of Fn3con (SEQ ID NO: 2) was derived from homologous fibronectin type III domains and optimised for protein stability.

Fn3con amino acid sequence
(SEQ ID NO: 2)
PSPPGNLRVTDVTSTSVTLSWEPPPGPITGYRVEYREAGGEWKEVTVPGS
ETSYTVTGLKPGTEYEFRVRAVNGAGEGPPSSVSVTT

FIG. 11 shows a sequence alignment between the polypeptide scaffold of the first aspect and Fn3 based polypeptides known in the art.

### Example 2: Protein Expression and Purification

Genes encoding FN3con and FnFN8, containing an N-terminal 6×HIS tag, followed by a thrombin cleavage site (LVPRGS), were chemically synthesized and provided in a pJexpress 404 plasmid by DNA2.0. The resulting plasmids were transformed into competent C41 E. coli cells for expression. A single colony from each transformation was picked and grown overnight at 37° C. in 200 ml of 2×YT (16.0 g/L tryptone, 10.0 g/L yeast extract, 5.0 g/L NaCl) media containing 100 μg/ml of ampicillin. These cultures were then used to seed 2 L of 2×YT media for the FN3con and FNfn8. Cultures were induced at an OD₆₀₀ of 0.9 with IPTG (0.5 mM final concentration), and grown for a further 5 hours at 37° C. The cultures were harvested and cell pellets resuspended in TBS (50 mM Tris, 150 mM NaCl, pH 7.4; EDTA free protease inhibitors, ThermoFisher), lysed via sonication and cellular debris removed by centrifugation (5,000×G). Recombinant protein was isolated from the whole cell lysate by metal affinity chromatography using loose NiNTA resin (Qiagen). Protein eluted from NiNTA resin was filtered (0.22 μm) then subjected to size exclusion chromatography using a Superdex 75 16/60 column (GE Healthcare) equilibrated in either PBS (140 mM NaCl, 2.7 mM KCl, 10 mM PO₄³⁻, pH 7.4) for biophysical characterization or low salt TBS (50 mM Tris, 50 mM NaCl, pH 7.4) for protein crystallography. Protein concentration was determined by Nanodrop ND-1000 (ThermoFisher) and protein was stored at 4° C. until use (biophysical characterization) or used immediately (protein crystallography).

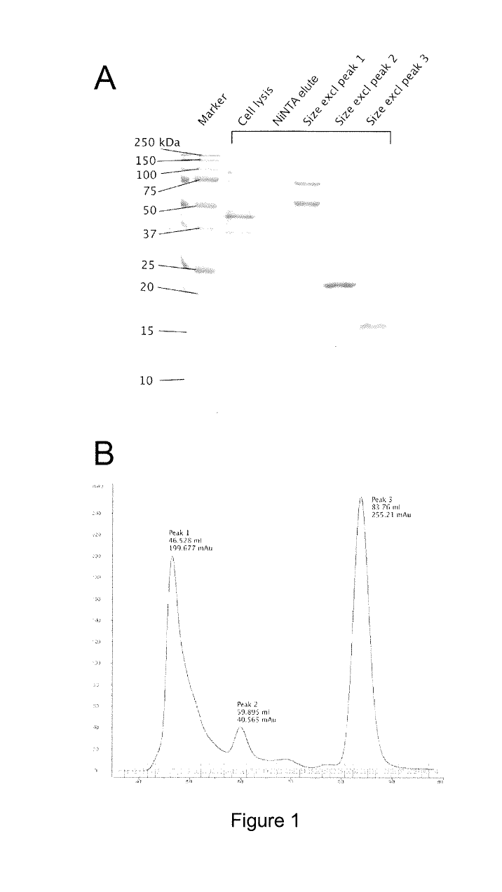

This process produced a homogenous, monomeric sample of the expected molecular weight (FIG. 1) that was further characterized by biophysical and X-ray crystallographic methods.

A set of well-studied FN3 domains (FNfn10, FNfn8 and TNfn3) and the consensus FN3 domains produced by Jacobs et al. (Fibcon and Tencon) were used for comparative analysis. All of these comparison domains have extensive biophysical data and X-ray crystal structures available and measured stabilities ranging from 57-90° C.

### Example 3: Characterisation of Thermal Stability

Thermal stability of purified FN3con and FnFN8 was measured by monitoring the circular dichroism (CD) signal at 222 nm to assess secondary structure content. Protein samples were used at a concentration of 0.2 mg/ml. Tₘ values were measured using a Jasco J-815 CD spectrophotometer with a peltier thermal control unit (CDF-426S). A quartz cuvette with a path length of 1 mm was used throughout. Samples were heated from 20° C. to 110° C. at a rate of 1° C. per minute and monitored at 222 nm. Far-UV scans from 195 nm and 260 nm were collected in triplicate at 20° C. before and after each melt. A buffer-only scan was collected in order to calculate a baseline. Following baseline removal, data was collected in triplicate, averaged and fit to a two-state unfolding model using a non-linear least squares fitting algorithm (Santoro and Bolen, 1988). The melting temperature (TM) was calculated as the G/Mg ratio.

### Equilibrium Measurements

A 6 M solution of guanidine isothiocyantate (GITC) in TBS was combined in varying ratios with TBS buffer using a liquid handling robot to create a range of denaturant solutions from 0-6 M GITC. These solutions were subsequently mixed in an 8:1 ratio with 9 μM protein in TBS to give a final concentration of 1 μM protein. All solutions were left to equilibrate at 25° C. for at least three hours, after which the fluorescence of each solution was measured on a Perkin Elmer LS55 fluorimeter using an excitation wavelength of 280 nm and an emission range of 300-400 nm. Readings were obtained from a 1 cm pathlength cuvette maintained at 25+/−0.1° C. The experiment was repeated, but using 9 μM protein pre-unfolded in 5 M GITC to generate a refolding curve. These solutions were left to equilibrate for at least six hours before their fluorescence was ascertained.

### Kinetic Measurements

Folding was monitored by changes in fluorescence using a 350 nm cut-off filter and an excitation wavelength of 280 nm. All experiments were performed using an Applied Photophysics (Leatherhead, UK) stopped-flow apparatus maintained at 25+/−0.1° C. For unfolding experiments, one volume of 11 μM protein solution was mixed rapidly with ten volumes of a concentrated GITC solution. For refolding, one volume of denatured protein in 4 M GITC solution was mixed with ten volumes of low-concentration GITC. In all cases, both solutions contained TBS buffer and were equilibrated at 25° C. for at least 30 minutes before use. Data collected from at least six experiments were averaged and traces were fit to a single or double exponential function as appropriate. Due to mixing effects, data collected in the first 2.5 ms were always removed before fitting.

### Data Analysis of Equilibrium and Kinetic Measurements

An Excel spreadsheet was used to derive the fluorescence average emission wavelength (AEW) for each of the equilibrated denaturant solutions (Royer et al., 1993). Excel was also used to convert each denaturant concentration into a denaturant activity since the two values are not directly proportional for GITC (Cota and Clarke, 2000). A plot of AEW against denaturant activity (Kaleidagraph, Synergy Software) yielded the expected sigmoidal plot, which was fitted to the standard two-state equation (Clarke and Fersht, 1993) to obtain the m-value (ΔG_{D-N}), the denaturant activity 50% ([D′]₅₀) and hence the stability of the protein in TBS buffer (ΔG_{D-N}). Both the unfolding and refolding AEW curves can be converted to Fraction Folded by first removing the baselines and then normalizing the resulting data.

All kinetic traces fitted well to a single exponential decay plus a linear drift term. Longer experiments indicated the presence of a much slower second refolding rate that was incompatible with the timescale of the stopped-flow apparatus. Since FN3con has 11 proline residues, we attribute this rate to proline isomerization although further experiments are required to confirm this hypothesis. An amplitude analysis suggests that this slower rate accounts for between 50% and 80% of all proteins at low concentrations of denaturant. The resulting chevron plot (Cota and Clarke, 2000) showed rollover in the refolding arm (indicating the presence of a refolding intermediate) and a kink in the unfolding arm (indicating the presence of a high energy intermediate). It was fitted using Prism (Synergy Software) to the following equation to estimate all parameters:

$$ln ( {k}_{obs} ) = ln ( \frac{1}{2} ( - {A}_{1} - \sqrt{{A}_{1}^{2} - 4 {A}_{2}} ) )$$

### Where:

$${A}_{1} = - ( {k}_{f} + {k}_{- 1} {e}^{{m}_{- 1} [ {D}^{′} ]} + {k}_{2} {e}^{- {m}_{2} [ {D}^{′} ]} + {k}_{- 2} {e}^{{m}_{- 2} [ {D}^{′} ]} )$$

$${A}_{2} = ( {k}_{f} ( {k}_{2} {e}^{- {m}_{2} [ {D}^{′} ]} + {k}_{- 2} {e}^{m - 2 [ {D}^{′} ]} ) + {k}_{- 1} {e}^{{m}_{- 1} [ {D}^{′} ]} + {k}_{2} {e}^{- {m}_{2} [ {D}^{′} ]} )$$

$${k}_{f} = {k}_{i} {e}^{- {m}_{i} [ {D}^{′} ]} ( \frac{1}{1 + \frac{{k}_{i} {e}^{- {m}_{i} [ {D}^{′} ]}}{{k}_{d} {e}^{- {m}_{d} [ {D}^{′} ]}}} )$$

ki and mi are the folding rate constant from the refolding intermediate (I) to the first transition state (TS1) and its associated m-value, k_{d} and an_{d} are from the denatured state (D) to the first transition state (TS1), k₋₁ and m₋₁ are unfolding from the high energy intermediate (I*) over TS1, k₂ and m₂ are folding from the high energy intermediate (I*) over TS2, k₋₂ and m₋₂ are unfolding from the native state (N) over TS2. By convention, k₋₁ is set as 100,000 s⁻¹ and m₋₁ is set as 0 M⁻¹: m2 is thus the m-value between TS1 and TS2 while the ratio k₋₁/k₂ informs on the difference in free energy between the two transition states.

### Results:

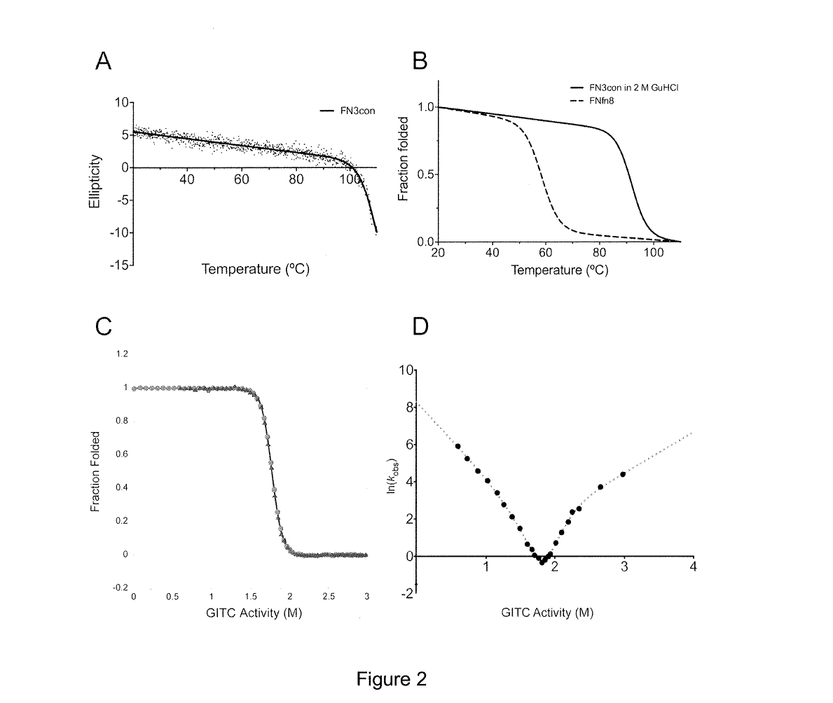

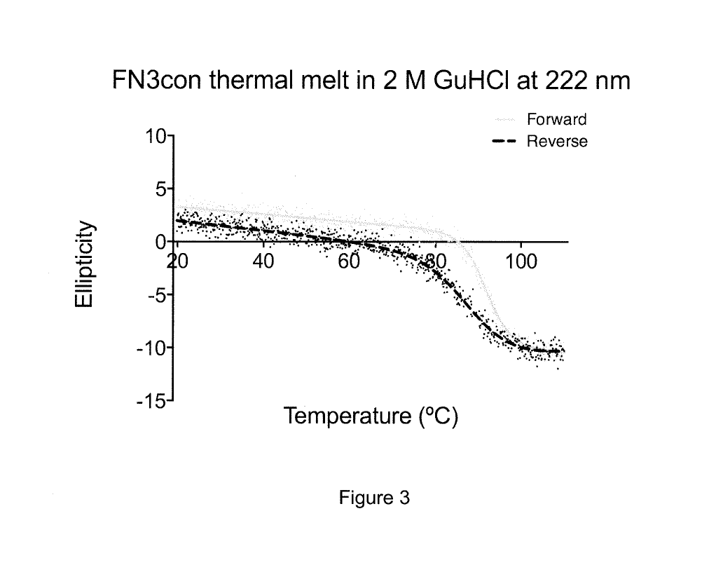

FN3con gradually loses secondary structure signal until about 100° C., where a sharp unfolding transition starts but does not plateau before the thermal limit of the CD spectrophotometer is reached (110° C.; FIG. 2A). We repeated the experiment in the presence of 2 M guanidine hydrochloride (GuHCl), which resulted in a complete unfolding transition and melting temperature (Tₘ) of 90.72° C. (FIG. 2B). Furthermore, we found FN3con to be reversibly foldable (FIG. 3), a common trait of the FN3 domain, and for comparison, we measured the Tₘ of FNfn8 to be 58.02° C. (FIG. 2B).

The unfolding and refolding equilibrium curves of FN3con show excellent agreement with one another, further indicating that the folding is reversible (FIG. 2C). The global fit to both datasets gives a denaturant activity midpoint, [D′]₅₀, of 1.75±0.01 M, an equilibrium m-value, m_{D-N}, of 8.80±0.21 kcal mol⁻¹ M⁻¹, and hence a protein stability, ΔG_{D-N}, of 15.5±0.4 kcal mol⁻¹. Note that these errors are those of the fit, and not the true errors of experimental replication. The m-value for FN3con (8.80 kcal mol⁻¹ M⁻¹) is in the range expected for homologous FN3 domains (6.38 and 9.42 kcal mol⁻¹ M⁻¹ for FNfn10 and TNfn3 in guanidine isothiocyanate (GITC) respectively) (Cota and Clarke, 2000). However, it is clear that FN3con is far more stable (15.5 compared with 9.38 and 6.68 kcal mol⁻¹). The kinetic chevron (FIG. 2D) can be fitted extremely well using a modified equation to take into account both a refolding intermediate and a high-energy intermediate. The [D′]₅₀ from the chevron is 1.80+0.05 M, which is identical to the value obtained from the equilibrium studies and is strong evidence that both experiments are measuring the global unfolding of the protein domain and not a local effect. The fit gives a kinetic m-value of 10.2±0.9 kcal mol⁻¹ M⁻¹, and a stability in buffer of 18.6±1.6 kcal mol⁻¹ M⁻¹. Again, the errors are of the fit, and not the true errors of experimental replication. Taken together, the equilibrium and kinetic folding data indicate that, while FN3con is similar in structure to natural FN3 domains (based on the m-values) it is at least twice as stable. This increase in stability is predominantly due to a much slower unfolding rate, although the domain also has a slightly faster folding rate (see Table I).

TABLE I
Summary of stability, equilibrium and kinetic measurements for FN3 domains

| | | ΔG (kcal | Folding | Unfolding | |
|---|---|---|---|---|---|
| Construct | Tₘ (° C.) | mol⁻¹⁾ | rate (s⁻¹) | rate (s⁻¹) | Source |
| FN3con | >100.0 | 15.5 ± 0.4 | 4020 | 3.09 × 10⁻⁸ | |
| Fibcon | 89.6 | 11.4 ± 1.5 | N/A | N/A | (Jacobs et al., 2012) |
| FNfn10 | 82.5 | 9.38 ± 0.13 | 240 | 2.3 × 10⁻⁴ | (Cota and Clarke, 2000) |
| Tencon | 78.0 | 10.6 ± 0.9 | N/A | N/A | (Jacobs et al., 2012) |
| FNfn8 | 58.0 | N/A | N/A | N/A | |
| TNfn3 | 57.1 | 6.68 ± 0.18 | 6.2 | 4.8 × 10⁻⁴ | (Hamill et al., 1998) |

### Example 4: Crystallization of FN3con

FN3con was purified in 50 mM Tris pH 7.4, 50 mM NaCl and was concentrated to 25 mg/ml (Milipore 3 kDa cutoff concentrator). Concentrated FN3con was filtered through a 0.22 μm centrifugal filter and crystals were obtained from 0.1M phosphate-citrate pH 4.2 and 40% PEG300 (JCSG+Suite, Qiagen). Drops were prepared 1:1 in 1 μl. Small hexagonal or cubic crystals were formed within three days.

### X-Ray Diffraction, Structure Determination and Refinement

FN3con crystals were flash frozen in liquid nitrogen without further cryoprotection. Diffraction data was collected at the Australian Synchrotron on the MX1 beamline. Diffraction data to 1.98 Å resolution was collected and processed with iMOSFLM (Battye et al., 2011). Complete data collection statistics can be found in Table II. The FN3con structure was determined by molecular replacement (MR) with Phaser (McCoy et al., 2007) using PDB entry 2CK2 (Ng et al., 2007) as a search probe (following removal of solvent atoms and trimming of side chains to create a poly-Ala model). The asymmetric unit contains one protein molecule. Model building and structure refinement was carried out with PHENIX v. 1.8.4-1496 (Adams et al., 2010) and Coot (Emsley and Cowtan, 2004). Coordinates of FN3con were deposited in the RCSB Protein Data Bank with PDB ID 4U3H.

TABLE II
Crystallographic data and refinement statistics

| | FN3con (4U3H) |
|---|---|
| Data collection | |
| Temperature | 100 K |
| X-ray source | Australian Synchrotron MX1 |
| Detector | ADSC Quantum 210R |
| Wavelength (Å) | 0.9537 |
| Space group | P 4₁ 3 2 |
| Unit cell axes (Å) | 86.1, 86.1, 86.1 |
| Angles (°) | 90, 90, 90 |
| Mol./ASU | 1 |
| Resolution (Å)^{a} | 35.15-1.98 (2.05-1.98) |
| Total reflections^{a} | 80,030 (95,144) |
| Unique reflections^{a} | 8,078 (804) |
| Completeness (%)^{a} | 100.0 (100.0) |
| Multiplicity^{a} | 35.3 (33.5) |
| Rₚᵢₘ^{a} | 0.018 (0.138) |
| <I/σI>^{a} | 28.5 (5.33) |
| CC1/2^{a} | 1.0 (0.935) |
| Structure refinement | |
| Resolution (Å) | 35.15-1.98 |
| Number of non-hydrogen atoms | 801 |
| Number of solvent molecules | 61 |
| R_{work} (%) | 0.1970 |
| R_{free} (%)^{b} | 0.2432 |
| RMSD bond lengths (Å) | 0.013 |
| RMSD bond angles (°) | 1.37 |
| Ramachandran plot | |
| % favoured (% outliers) | 100.00 (0.0) |
| Clashscore | 0.7 |
| MolProbity score | 0.73 100^{th} percentile* |
| | (N = 12332, 1.980 Å ± 0.25 Å) |

^{a}Values for highest resolution shell are in brackets.
^{b}The free R factor was calculated with 5% of data omitted from refinement.
*100th percentile is the best among structures of comparable resolution; 0th percentile is the worst.

### Structure Analysis

In analysis of FN3con, residue numbering was kept as per amino acid positions of the construct, due to non-ideal sequence to structure alignment with the other FN3 domains. In analysis of FNfn8, residues 1238-1325 in 1FNF were renumbered 1-88 with residue P1238 as residue 1. In analysis of FNfn10, residues 1416-1509 of 1FNF were renumbered 1-94 with residue V1416 as residue 1. In analysis of TNfn3, residues 802 to 891 from the original PDB file of 1TEN were renumbered 1-90 with residue R802 as residue 1. In analysis of Fibcon and Tencon, residue numbering was unchanged and is per respective PDB files 3TEU and 3TES. C-terminal His tags from Fibcon and Tencon were removed for structural analysis and molecular dynamics simulations.

Structural alignments were performed using the Mustang-MR webserver (Konagurthu et al., 2010). H-bonds and salt-bridges (<7 Å) were calculated using the WHATIF server (Hekkelman et al., 2010). Accessible surface area (ASA) was calculated using the ASA tool from CCP4 (Winn et al., 2011). The grand average hydropathy (GRAVY) score was calculated using the ProtParam tool provided byExPASy and uses the Kyte and Doolittle hydropathy value for each amino acid (Kyte and Doolittle, 1982). Total cavity volume was calculated using the CASTp web server (Dundas et al., 2006) using a 1.4 Å probe radius. Mean occluded surface packing (OSP) was calculated using the OS software (Fleming and Richards, 2000).

### Results:

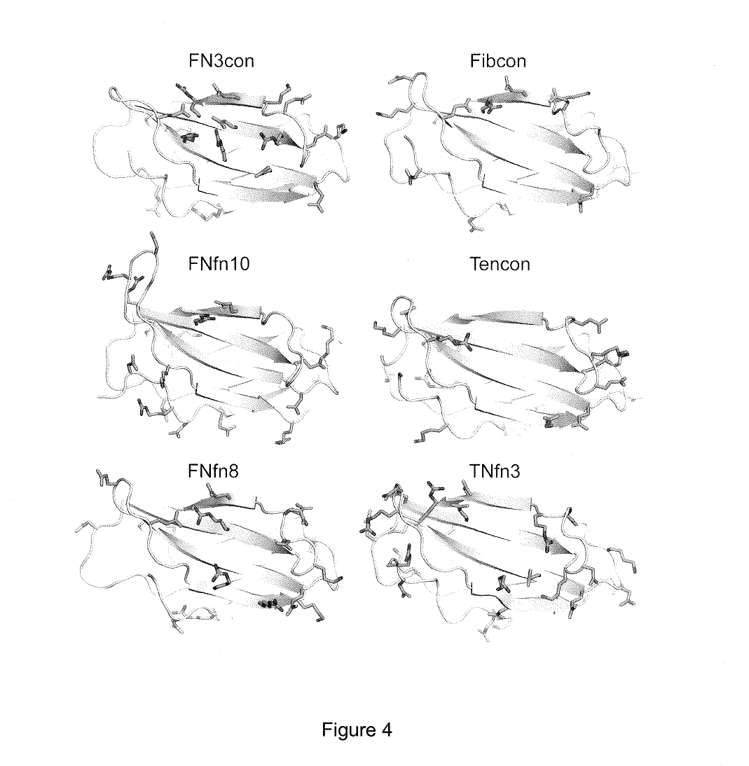

FN3con adopts the FN3 fold, consisting of 7 anti-parallel β-strands connected by surface exposed loops (FIG. 4). A structural alignment with our comparison domains shows very high similarity, with an average root mean square deviation (RMSD) of 1.2 Å across backbone Ca atoms in all structures.

TABLE III
Global analysis of molecular contacts.

| | | | | Salt | | | | |
|---|---|---|---|---|---|---|---|---|
| | | | | bridge | | | Negative | Positive |
| | PDB | Tₘ | H- | interactions | | GRAVY | charged | charged |
| Construct | code | (° C.) | bonds^{a} | <7 Å^{a} | ASA (Å²)^{b} | score^{c} | residues^{d} | residues^{d} |
| FN3con | 4U3H | >100 | 46 | 48 | 4545.5 | −0.536 | 10 | 7 |
| Fibcon | 3TEU | 89.6 | 42 | 14 | 4882.3 | −0.190 | 8 | 3 |
| FNfn10 | 1FNF | 82.5 | 41 | 9 | 5470.8 | −0.115 | 8 | 8 |
| Tencon | 3TES | 78.0 | 44 | 28 | 5093.3 | −0.307 | 12 | 7 |
| FNfn8 | 1FNF | 58.02 | 43 | 28 | 5239.0 | −0.377 | 11 | 7 |
| TNfn3 | 1TEN | 57.1 | 45 | 41 | 5163.5 | −0.577 | 18 | 9 |

^{a}Hydrogen bonds and salt bridges were calculated using the WHATIF server (Hekkelman et al., 2010).
^{b}Calculated using the ASA tool from ccp4 (Winn et al., 2011).
^{c}Calculated using the ProtParam tool provided by ExPASy and uses the Kyte and Doolittle hydropathy value for each amino acid (Kyte and Doolittle, 1982).
^{d}Negatively charged amino acids are counted as Asp and Glu, whilst positively charged amino acids are counted as Arg and Lys.

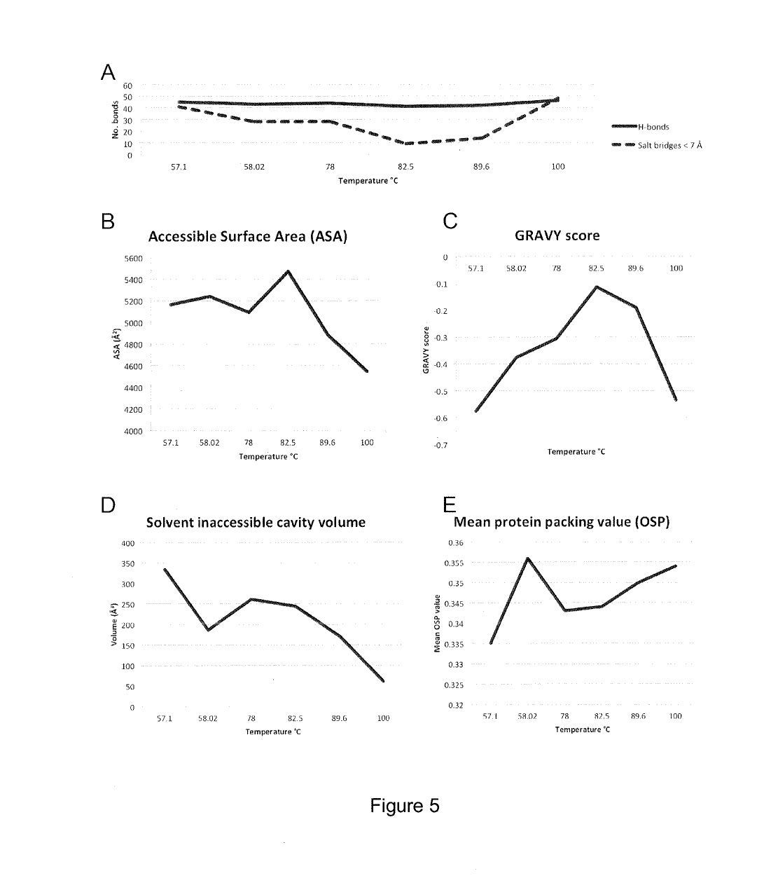

To investigate the structural basis for increased stability in FN3con, we first calculated several physicochemical and structural parameters that are known to affect protein stability and folding, for the set of comparison domains (Table III). Analysis reveals FN3con to have the highest count of H-bonds (46) and salt bridges (48), with the smallest accessible surface area (ASA). Comparatively, the count of H-bonds is relatively equal across the assessed domains, with a mean count of 43.5. Salt bridge counts are highly varied across the comparison set. Although FN3con has the highest number of salt bridges (48), consistent with its high stability, TNfn3 (lowest stability) has the second highest count with 41 salt bridges. However, comparisons with the ratio of acidic:basic residues show large differences between FN3con and TNfn3. Specifically, FN3con has 48 salt bridges being formed from 10 positive and 7 negatively charged residues, whilst TNfn3 has 41 salt bridges being formed by 18 positive and 9 negatively charged residues. This data alone suggests a significant charge mismatch and potential clashes. When taken in context of the structure, FN3con reveals a unique and extensive complementary charged electrostatic network that is distributed over β-sheet 2, spanning strands C′, C and F. This network results from the presence of four arginine residues and four glutamic acid residues (R45, R49, R81, R83, and E47, E57, E79, E90), which are not present in any of the other FN3 domains (FIG. 4). Comparatively, TNfn3 reveals clustering of like-charged residues on the peripheral loops (FIG. 4). Calculations of ASA values correlate weakly to thermal stability, with FN3con and Fibcon having the smallest ASA values of 4545.5 Å² and 4882.3 Å² and the highest thermal stability, however this trend does not appear to be linear for the remaining domains. Similarly, the grand average hydropathicity (GRAVY) scores vary quite dramatically across the set of comparison domains and do not appear to correlate with thermal stability (FIG. 5A-E).

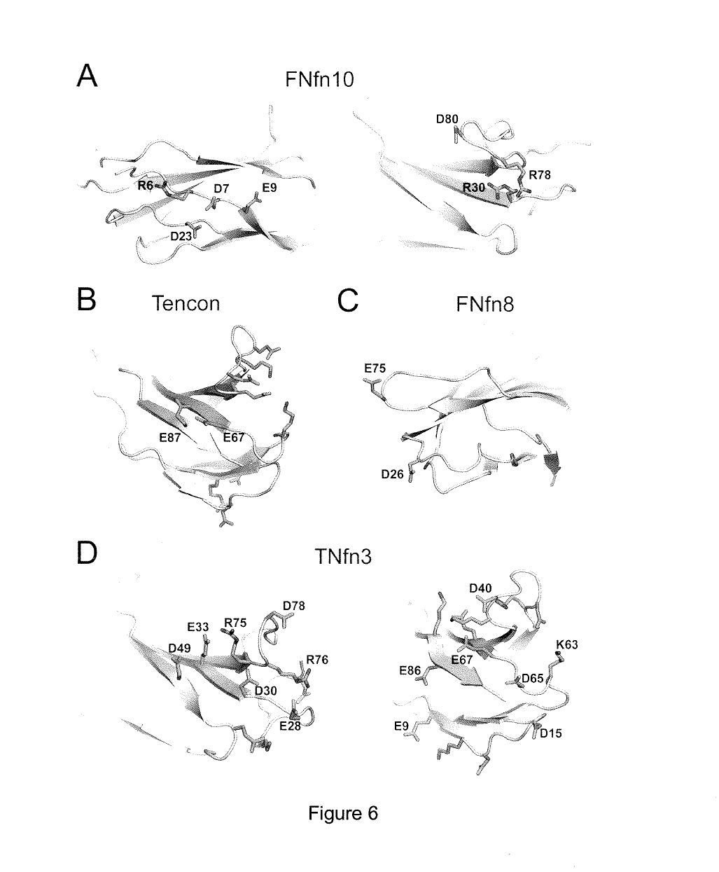

Whilst salt bridge interactions are thought to make a relatively minor contribution to stability, the presence of unfavorable clusters with like-charged residues are known to be destabilizing and may offer clues to the differences in stabilities of the assessed FN3 domains. Indeed, such like-charged clusters are present in the metastable FN3 domains (FNfn10, Tencon, FNfn8 and TNfn3) but absent in the highly stable FN3con and Fibcon (FIGS. 4 and 6). This is clearly seen in FNfn10, which features both negatively (D7, E9 and D23) and positively (R30, R78 and D80) charged clusters (FIG. 6A). The destabilizing effect of the first cluster has been validated by mutagenesis, where mutation of D7 to asparagine or lysine increased thermal stability by ˜10° C. at pH 7.0. Similarly, potentially destabilizing clusters are also present in Tencon (E67 and E87) (FIG. 6B), FNfn8 (D26 and D52) (FIG. 6C) and TNfn3 (E33 and D49; E28, D30 and D78; E9 and E8; D15 and D65; D40 and E67) (FIG. 6D). Unsurprisingly, there is a strong similarity in the distribution of charged residues amongst Tencon and TNfn3. However, Tencon appears to have reduced the presence of like-charged residue clusters, resulting in increased coordination of complementary charged residues (FIG. 4). When taken into a structural context, we hypothesize that the introduction of a unique salt bridge network in FN3con and lack of unfavorable like-charged residue clusters may greatly influence the experimentally observed slow unfolding rate.

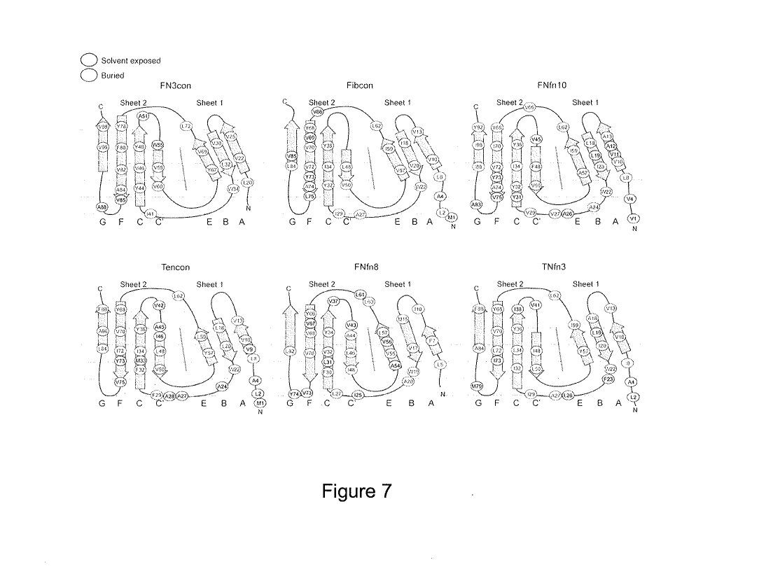

The hydrophobic effect is a major determinant of protein folding and stability. We therefore assessed differences in hydrophobic packing amongst the comparison set of FN3 domains, focusing on a hydrophobic “banding” pattern that is orthogonal to the direction of the β-strands (FIG. 7). Strikingly, the degree of uniformity and alignment amongst hydrophobic residues in each band appears to be proportional to the stability of the domain. In general, we observe higher stability to be associated with uniform hydrophobic banding as well as greater burial and reduction of bulky hydrophobic residues, which is in line with the current understanding of the hydrophobic effect and its role in stability (FIG. 7).

As packing density of the hydrophobic core is a known factor in protein stability and since the FN3 fold is characterized by two β-sheets with a tertiary arrangement around a hydrophobic core, we calculated the volumes of solvent inaccessible cavities and the mean occluded surface packing (OSP) value for each FN3 domain, as a measure of packing density (Table IV). The most striking observation from these calculations is the significantly reduced solvent inaccessible cavity volume of FN3con (60.8 Å³) compared to the next best domain, Fibcon (171.0 Å³) (Table IV). This value alone indicates superior packing of the hydrophobic core in FN3con and may contribute to the observed fast folding rate. Interestingly, FNfn8 is measured to have a cavity volume of 185.8 Å³, suggesting that whilst cavity volume may be an indicator of stability, it is by no means absolute. A similar anomaly was also seen for a chimera of FNfn10 and TnFN3, which had a stability that was intermediate between the two proteins, despite having a core that was less well packed than either parent. The results from the OSP calculations (Table IV) show FN3con and Fibcon to have one of the highest OSP values (0.354 and 0.350 respectively), however, according to this metric, their surface is slightly less packed than FNfn8 (0.356). This further suggests a complex and mostly non-linear accumulation of context dependent properties that govern protein stability (FIG. 5).

TABLE IV
Packing densities of FN3 domains.

| | | Total cavity | Mean protein packing |
|---|---|---|---|
| Protein | Tₘ (° C.) | volume (Å³)^{a} | value (OSP)^{b} |
| FN3con | >100 | 60.81 | 0.354 |
| Fibcon | 89.6 | 171.0 | 0.350 |
| FNfn10 | 82.5 | 243.9 | 0.344 |
| Tencon | 78.0 | 260.7 | 0.343 |
| FNfn8 | 58.02 | 185.8 | 0.356 |
| TNfn3 | 57.1 | 334.1 | 0.335 |

^{a}Calculated using the CASTp web server (Dundas et al., 2006) with a 1.4 Å probe radius.
^{b}Calculated using the OS software (Fleming and Richards, 2000); a higher value indicates better packing.

We next investigated the structural context of aromatic residues, which are known to contribute greatly to the stability of immunoglobulin-like domains. Analysis reveals that all assessed FN3 domains contain the highly conserved tryptophan 22 (W22), whilst FN3con further contains a unique solvent exposed tryptophan (W55) on β-sheet2 (FIG. 7). W55 is seen to pack tightly against the side chains of E47, R49, E79 and R81, however its role is not immediately apparent. Tyrosine residues are another highly conserved motif amongst the immunoglobulin fold and are thought to contribute to stability via the concept of a ‘tyrosine corner’. In particular, tyrosine corners feature tyrosine residues positioned near the beginning or end of an antiparallel β-strand. Tyrosine corners in the FN3 superfamily are involved in early structure formation and are thus important for stability of the structure, with tyrosine to phenylalanine mutations costing 1.5 to 3 kcal mol⁻¹ in stability. Subsequently, they are thought to stabilize the structure by playing an amphipathic role, in which the hydroxyl group points outwards to the solvent and can mediate interactions with water whilst the phenyl ring can simultaneously engage in stabilizing hydrophobic interactions. Comparisons amongst the selected FN3 domains reveal two highly conserved tyrosine residues, one at the N-terminal end of strand C (Y48 in FN3con, Y36 or Y34 in others) and the other at the C-terminal end of strand F (Y78 in FN3con, Y68 or Y66 in others) (FIGS. 7 and 8). The higher stability FN3 domains of FN3con, Fibcon and TNfnl 0, contain a tyrosine residue at the C-terminal end of strand C (Y44 in FN3con and Y32 in Fibcon and FNfn10), potentially providing stabilizing interactions to both loop regions, which is absent in the lower stability domains. Interestingly, FN3con, Tencon and TNfn3 share a unique tyrosine residue (Y67 and Y57 respectively) on β-sheet 1, which is absent in Fibcon, FNfn10 and FNfn8 (FIGS. 7 and 8).

### Example 4: Molecular Dynamics Simulations

Simulations of FN3con, Fibcon, FNfn10, Tencon, FNfn8 and TNfn3 were based on the following crystal structures with PDB codes 4U3H, 3TEU, 1FNF, 3TES, 1FNF, 1TEN respectively. Coordinates were prepared by removal of crystal waters, N- or C-terminal His tags and extracted from their respective PDB files as per listings in the Structure analysis methods section. Residues with missing atoms were modelled using MODELLER (Eswar et al., 2007), followed by capping of the N- and C-termini with the neutral N-methyl amide and acetyl groups. All residues were simulated at their dominant protonation state at pH 7. Completed structures were solvated in a cubical simulation box with a minimum distance of 1.4 nm from any protein atoms to the box wall, followed by the addition of sodium and chloride ions to neutralize the system. Extra NaCl was added to reach a final concentration of approximately 150 mM NaCl. System dimensions and compositions are listed in Table V.

TABLE V
Simulation system dimensions and composition

| | | Sodium | Chloride | Water | Total atoms |
|---|---|---|---|---|---|
| System | Dimensions | ions | ions | molecule | (approximate) |
| FN3con | 6.8 nm3 | 32 | 29 | 10,262 | 31,700 |
| Fibcon | 7.3 nm3 | 40 | 35 | 12,313 | 37,800 |
| FNfn10 | 7.0 nm3 | 31 | 31 | 10,888 | 33,600 |
| Tencon | 7.0 nm3 | 36 | 31 | 11,019 | 31,700 |
| FNfn8 | 7.1 nm3 | 15 | 11 | 11,600 | 35,700 |
| TNfn3 | 7.2 nm3 | 44 | 34 | 11,905 | 36,700 |

### Simulation Protocol

All simulation systems were subjected to energy minimization, followed by equilibration in the NPT ensemble (26.85° C. (300 K), 1 bar (˜1 atm)) or (94.85° C. (368 K), 1 bar (˜1 atm)), with 1,000 kJ mol⁻¹ nm⁻² positional restraints applied to all non-hydrogen atoms; restraints were stepped down 10 fold every 100 ps over 300 ps. Equilibrated systems were run at 300 K and 368 K for 1 μs and 2 μs, in triplicate, with each replicate starting from a different distribution of initial velocities. All simulations were performed using GROMACS ver 4.0.7 (Hess et al., 2008) in conjunction with the GROMOS 53A6 united-atom force field (Oostenbrink et al., 2004). Water was represented explicitly using the simple point-charge (SPC) model (Berendsen et al., 1981). All simulation systems were performed in an NPT ensemble under periodic conditions. Temperature was maintained close to its reference value of 300 K or 368 K by V-rescale temperature coupling (Berendsen et al., 1984). Pressure was maintained close to a reference value of 1 atm by isotropic coupling with a Berensden pressure bath (Berendsen et al., 1984). Non-bonded interactions were evaluated using a twin range cut-off scheme: interactions falling within the 0.8 nm short-range cutoff were calculated every 2 fs whereas interactions within the 1.4 nm long cutoff were updated every 10 fs, together with the pair list. A generalized reaction-field correction was applied to the electrostatic interactions beyond the long-range cutoff (Tironi et al., 1995), using a relative dielectric permittivity constant of εRE=62 as appropriate for SPC water (Heinz et al., 2001). All bond lengths to hydrogen atoms were constrained using the P-LINCS algorithm (Hess et al., 1997) and water geometry was constrained using the SETTLE algorithm (Miyamoto and Kollman, 1992). A leap-frog integrator (Hess et al., 2008) was used throughout, with a time step of 2 fs.

### Simulation Analysis

Analyses of the simulations were performed using the tools provided in the GROMACS package 4.0.7 (Hess et al., 2008) and custom scripts in conjunction with ProDy (Bakan et al., 2011). Graphs and plots were produced using Matplotlib (Hunter, 2007). Molecular graphics were prepared with PyMol ver. 1.3.2 (DeLano, 2002) and Visual Molecular Dynamics (VMD) 1.9.2 (Humphrey et al., 1996).

### Results:

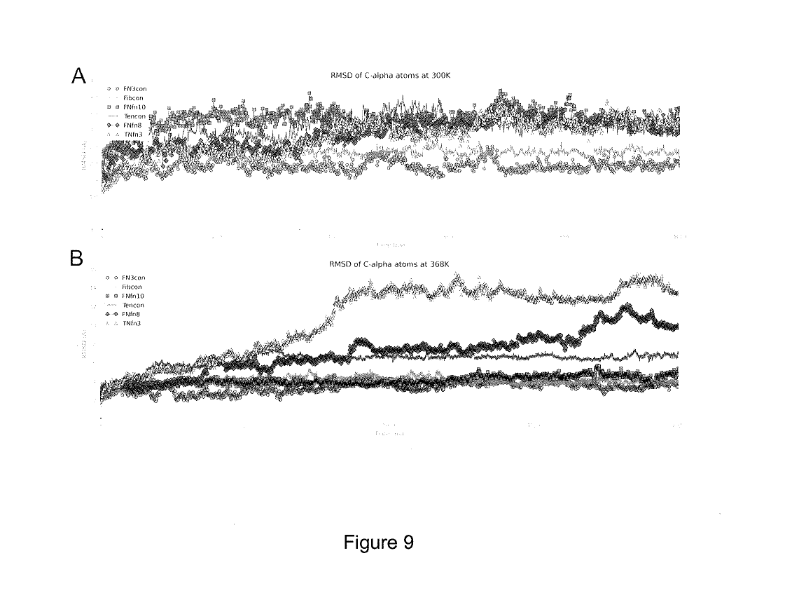

All domains display similar dynamic behavior at 300K, showing relatively low flexibility within the β-sheet and greater motions in the flexible loops, as expected (FIG. 9A). FN3con and Fibcon are both slightly more rigid than FNfn10, Tencon, FNfn8 and TNfn3 at 300 K, however at 368K dramatic differences are evident. At 368 K, FN3con, Fibcon and FNfn10 remain folded, with an average RMSD of 3.4 Å, 4.1 Å and 4.1 Å respectively. Comparatively, FNfn8 and TNfn3 start to unfold after 500 ns, with unfolding essentially complete after 1 μs, whilst Tencon shows signs of partial unfolding in some of the replicates at approximately 500 ns (FIG. 9B). Strikingly, the MD simulations faithfully support the experimentally derived stability hierarchy (FIG. 9B and Table I).

### Strand Swapping May Play a Role in Thermostability and Unfolding

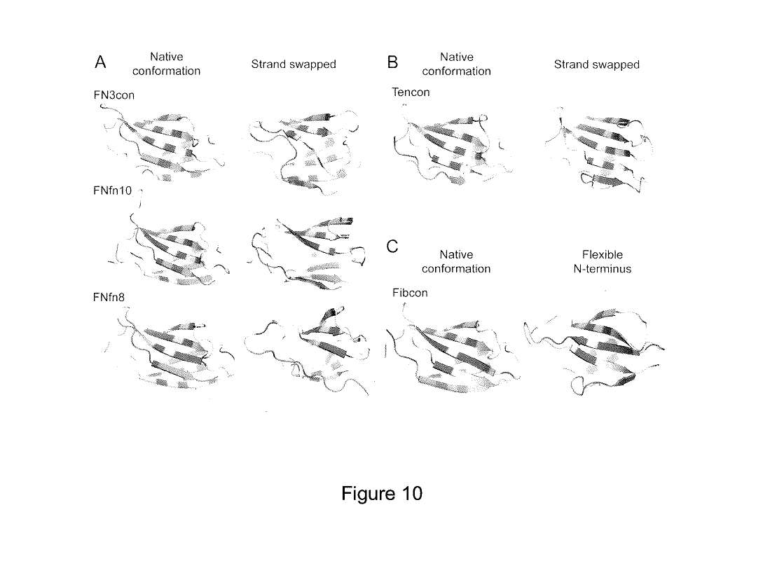

Analysis of the simulation trajectories at 368 K reveals that, with the exception of Fibcon and TNfn3, all domains reveal some degree of either the N- or C-terminal strand swapping from one sheet to the other. Specifically, in FN3con, FNfn10 and FNfn8, we observe strand G to swap from β-sheet 2 to 0-sheet 1 (FIG. 10A). Intriguingly, this is reversed in Tencon, with strand A swapping from β-sheet 1 to β-sheet 2, forming a 5 stranded β-sheet (FIG. 10B). The role of ‘strand swapping’ is not immediately obvious from the simulations. Strand swapping at 300 K is not observed over 1 μs, which may be due to a lack of conformational sampling. Therefore, it may be reasonable to suggest that strand swapping is an event that precedes or initiates the unfolding pathway by compromising the hydrophobic core. This is suggested by analysis of strand swapping in Tencon and FNfn8, in which both of these structures exhibit partial to full unfolding after strand swapping. Although no strand swapping is seen in the Fibcon simulations, we instead observe the N-terminal strand to undergo large structural rearrangements that may expose the hydrophobic core to solvent and lead to eventual unfolding (FIG. 10C). In the case of TNfn3, we do not observe any strand swapping, but rather, strands A and G of TNfn3 to pull closer together in concert, followed by rapid unfolding. This motion does not appear to directly initiate unfolding, which is rapid and cooperative in nature; however, it is difficult to ascertain if this is due to the simulation temperature being significantly higher than the measured melting temperature. Given the prevalent like-charged residue clusters in TNfn3, unfolding may instead be initiated by electrostatic repulsion at both peripheral loops (FIG. 6D).

### The Role of Electrostatics in FN3 Domain Dynamics

Structural comparisons of FN3 domains revealed contrasting electrostatic interactions likely to induce positive or negative effects on stability (FIGS. 4 and 6). We therefore investigated whether electrostatics also play a role in the dynamics of FN3 domains. The complementary electrostatic mesh on β-sheet 2 of FN3con (FIG. 4) is stable throughout the simulations (at 300 K and 368 K) and we suggest this to be a stabilizing factor during the stress of high temperature on the structure by contributing to slowing down the unfolding rate (FIG. 10A). In contrast, one of the few surface electrostatic interactions in Fibcon (involving E47, E80 and R33) is short-lived during the simulation at 300 K and 368 K and thus unlikely to make a large contribution to stability. During the simulations of FNfn10 at 368 K, the negatively charged cluster of D7, E9 and D23 is highly mobile, with charge repulsion causing the N-terminus to peel away into solvent and exposing the hydrophobic core. In addition, the neighboring positively charged residues R30 and R78 on strands C and F in FNfn10 rapidly rearrange throughout the simulation, with R30 burying itself into the hydrophobic core. In the Tencon structural analysis, we predicted charge repulsion of E67 and E87. The resulting dynamics simulations suggest this to have some impact on the dynamics of the local area, with strands C and F regularly peeling away from one another at the E/F and C/C′ loop peripheries. Finally, in FNfn8, the region surrounding residues D26 and E75 show pronounced motion prior to unfolding, suggesting a negative contribution to stability. Taken together these observations are indicative of and consistent with electrostatic residues playing an important role in protein dynamics, stability, and unfolding.

### Rigidity of Uniform Hydrophobic Core in FN3 Domains May Contribute to its Stability and Folding

The hydrophobic core of FN3con is highly regular, exhibiting uniform banding of hydrophobic residues (FIG. 7). Strikingly, this uniformity is retained throughout the high temperature simulations and after strand swapping, a phenomenon that also occurs in FNfn10 and Tencon (FIG. 10A). In particular, the uniformity of FN3con is due to residues V96 and V98 realigning with L20 and V22 in strand A as strand G swaps from β-sheet 2 to β-sheet 1.

### Dynamic Recruitment of Tyrosine Corner Residues

All of the assessed FN3 domains contain the highly conserved tyrosine residue, Y78 in FN3con and Y68/Y66 in the others. During the simulations of all domains at 368 K, position Y78 is observed to be capable of dynamic rearrangement during strand swapping and on thermal warping. Specifically, position Y78 is seen to be recruited from the C′/E solvent interface to mediate solvent interactions when strand F becomes slightly separated from strand C. Furthermore, the relatively stable domains of FN3con, Fibcon, and FNfn10 contain a conserved tyrosine corner (residues Y44, Y32 and Y32, respectively) (FIGS. 7 and 8). This residue is not present in the less stable domains of Tencon, FNfn8 and TNfn3. In the simulations of FN3con, Fibcon and FNfn10, the sidechains of Y44/Y32 are relatively rigid, suggesting a specialist role in stability that is consistent with other findings (Cota et al., 2000). In FNfn8, a tyrosine residue is not present in this position, and as such, high temperature simulations show that the solvent exposed Y74 in the G/F loop is recruited to fulfill this role; however, given its position in the structure, such recruitment appears to have a destabilizing effect in the local area. In Tencon, although Y73 is nearby, it is not positioned in the G/F loop, but rather at the C terminus of strand F; this positioning restricts dynamic motion and thus does not appear to play a role in stability. In TNfn3, there are no nearby tyrosine residues available to fulfill this role. As mentioned in the structural analysis (FIGS. 7 and 8), FN3con contains an additional tyrosine corner motif (Y67), whose interactions are almost identical to the equivalently positioned Y57 of Tencon and TNfn3, but absent in all other domains. In a previous MD simulation of TNfn3, Y36 makes several potentially stabilizing, non-crystallographic interactions (H-bonds and VdW) with Y57 and 120 (Paci et al., 2003), which may indicate that the equivalent Y67 of FN3con makes a similar contribution to stability. Our simulations of FN3con show long-lived conformations of Y67 and Y48, suggesting that they play a role in stabilizing the C/E strand solvent interface.

### DISCUSSION

In this study we have described the consensus design and subsequent biophysical, structural and dynamical characterization of a novel FN3 domain, FN3con.

Overall, FN3con is the most stable FN3 domain reported to date, having a Tₘ in excess of 100° C. and a ΔG_{D-N} of 15.5 kcal mol⁻¹. It folds reversibly via two-state kinetics, with relatively fast folding and very slow unfolding rates (FIG. 2, Table I, FIG. 3).

In an effort to determine the molecular basis of stability in FN3con, we determined its X-ray crystal structure, which allowed structural and dynamics analyses and comparisons with Fibcon, FNfn10, Tencon, FNfn8 and TNfn3. As such, our results reveal that the superior stability of FN3con originates from highly specific and optimized electrostatic and hydrophobic interactions, as well as dynamic adaptability of the hydrophobic core at high temperature.

Calculations of physiochemical properties from the crystal structures revealed no relationship in the number of hydrogen bonds to thermal stability. However, there is a distinct difference in the number of possible salt bridges. Intriguingly, FN3con is capable of 48 salt bridge interactions, whilst the least stable TNfn3 is capable of 41 salt bridge interactions—the second highest count in Table III. A closer inspection revealed that the ratio of positive and negatively charged residues reveals FN3con to have 48 salt bridges being formed from 10 positive and 7 negatively charged residues, whilst TNfn3 has 41 salt bridges being formed by 18 positive and 9 negatively charged residues; suggesting significant charge mismatches. In context of structure, we see significant differences in positioning of the charged residues. FN3con reveals a unique and extensive complementary charged electrostatic network that is distributed over β-sheet 2. This network consists of four arginine and four glutamic acid residues, and is not present in any of the other FN3 domains (FIG. 4). Comparatively, TNfn3 contains a cluster of like-charged residues on the peripheral loops, which are likely to be destabilizing (FIG. 4). The remaining FN3 domains show no sign of a linear correlation between salt bridge count and stability. This implies that stability is related to the context of salt bridge interactions rather than a numerical metric of potential interactions. The role of electrostatic interactions and their relation to thermal stability has been studied extensively. Surface electrostatic interactions typically make small contributions (˜0.5 kcal mol⁻¹) to the overall stability, and tend to be context dependent and non-additive in nature. The energetic contribution provided by the electrostatic mesh in FN3con would be challenging to assess, given that each charged residue influences each other over long distances (2-7 Å). Although surface charged residues are unlikely to play a major role in thermodynamic stability, they may influence kinetic stability via effects on folding and unfolding rates. Accordingly, we hypothesize that the complementary electrostatic network seen in FN3con contributes to the dramatic reduction in unfolding rate, which has been reported for some thermophilic proteins.

As the hydrophobic effect is known to be a major driver of protein folding and stability, we assessed differences in hydrophobic residues amongst the set of FN3 domains. Comparative analysis of hydrophobic residues reveals the presence of a banding pattern that is orthogonal to the direction of the β-strands (FIG. 7). This banding pattern is well known and important in formation of the folding nucleus. Strikingly, the degree of uniformity and alignment amongst hydrophobic residues in each band appears to be proportional to the stability of the domain. In general, we observe higher stability to be associated with uniform hydrophobic banding as well as greater burial and reduction of bulky hydrophobic residues, which is consistent with the established role of the hydrophobic effect and in protein stability (FIG. 7 and Table IV). One of the most striking observations from our physiochemical properties was the dramatic decrease of solvent inaccessible cavity volume in FN3con, which is 2.8× smaller than the next best structure, Fibcon (Table IV). As packing density of the hydrophobic core is a known factor in protein stability, we suspect this attribute plays a significant role in the observed fast folding rate of FN3con.

Structural analysis revealed the introduction of a cooperative electrostatic network, optimization of the hydrophobic core packing in FN3con and acquisition of tyrosine corner residues in a positional pattern that is not seen in any of the assessed FN3 domains. Given the complexity of interactions, we employed MD simulations to provide insight into the dynamics at ambient—(300 K) and high-temperature (368 K). Strikingly, the MD simulations at 368 K faithfully recapitulate the experimentally derived stability hierarchy (FIG. 9B and Table I). Overall, the simulation trajectories reveal partial unfolding of Tencon and loss of native structure in FNfn8 and TNfn3 around 500 ns, which we attribute to the start of an unfolding pathway. On closer inspection of the simulation trajectories, FN3con, FNfn10 and FNfn8 tend to have the C-terminal strand (strand G) to swap from β-sheet 2, to β-sheet 1, at high temperatures (FIG. 10A). Intriguingly, the simulations of Tencon reveal the N-terminal strand (strand A) to swap from β-sheet 1 to β-sheet 2 (FIG. 10B). The strand swapping of Tencon is dramatically different and forms a 5-stranded β-sheet, however, the role of strand swapping in stability is not immediately apparent from our simulations. Interestingly, mutations in the F/G loop of Tencon have been shown to promote strand-swapping of the C-terminal strand (strand G), as well as influencing the resulting aggregation properties; however, it is unclear as to how this relates to the dynamics observed at 368 K, especially since strand G remains stable throughout MD of Tencon. Although there exists only one example of strand swapping within the current FN3 literature, folding studies, including phi-value analysis, of FN3-like domains indicate folding occurs through a common-core ring involving strands B, C, E and F, leaving strands A and G to pack last. This therefore suggests a lack of constraints on strands A and G and supports the strand swapping events we observe during the high temperature simulations (FIG. 10). Subsequently, we hypothesize that strand swapping is an event on the unfolding pathway.

The high temperature simulations show that in FN3con and FNfn10, as strand G swaps from β-sheet 2 to β-sheet 1, the hydrophobic residues in strand G align perfectly to those in strand A (FIG. 10A). Subsequently, Tencon shows the ability to dynamically realign its hydrophobic residues during N-terminal strand swapping; forming a 5 stranded β-sheet on sheet 2 (FIG. 10B). Although FNfn8 undergoes strand swapping, the hydrophobic residues on strand G do not successfully align up with those in strand A (FIG. 10A), suggesting that the ability to realign the hydrophobic residues in strands A and G at elevated temperature plays a role in stability; however the exact role in either folding or unfolding is not apparent from our data.

MD simulations at 368 K reveal flexibility of loop regions in all structures, providing cavities for solvent to enter and potentially destabilise the hydrophobic core. Tyrosine corners feature tyrosine residues positioned near the beginning or end of an antiparallel β-strand. This feature is highly conserved, ubiquitous and exclusive to Greek key proteins. Tyrosine corners in the FN3 superfamily are involved in early structure formation and are important for stability of the structure, with tyrosine to phenylalanine mutations costing 1.5 to 3 kcal mol⁻¹ in stability. Our analysis of tyrosine residues showed a striking trend in that the most stable FN3 domains (FN3con, Fibcon and FNfn10) all contain tyrosine corners evenly spread throughout their structures and accessible to both peripheral loop regions.

Specifically, FN3con, Fibcon and FNfn10 make use of a unique tyrosine residue (Y44, Y32 and Y32 respectively) at the C-terminal end of strand C; a trait not observed in Tencon, FNfn8 and TNfn3 (FIGS. 7 and 8). Intriguingly, FN3con, Tencon and TNfn3 share a unique tyrosine residue (Y67, Y57 and Y57 respectively) at the C-terminal end of strand E in sheet 1 (FIGS. 7 and 8). The position of Y57 is suggested to play a small contribution to stability in TNfn3 by forming H-bond and Van der Waals interactions to Y36 (Paci et al., 2003). Given a similar environment in FN3con, we predict a similar contribution to stability in FN3con; and dynamics subsequently showed rigidity of these two residues. In addition, simulations at 368 K reveal the capacity for rearrangement and recruitment of tyrosine residues as the structures move at high temperature. One of the most striking differences is the lack of Y44/Y32 in Tencon, FNfn8 and TNfn3. Although FNfn8 attempts to recruit the solvent-exposed Y74, which is similarly positioned to Y44/Y32, it appears to result in destabilizing the local area (FIG. 9). Furthermore, Tencon and TNfn3 lack the ability to reposition a tyrosine residue to this region. As such, we hypothesize that the presence of a unique distribution of tyrosine corners in FN3con provides stabilizing features and may be capable of contributing to the observed slow unfolding rate.

In conclusion, we have successfully generated an FN3 domain, FN3con, which has unprecedented stability, with experimental data highlighting a Tₘ in excess of 100° C., a ΔG_{D-N} of 15.5 kcal mol⁻¹, reversible folding via two-state kinetics, with the fastest folding and slowest unfolding rates reported to date. Structural and dynamical analysis reveals that FN3con stability does not result from a single mechanism, but rather the combination of several features and a strong tendency to remove non-conserved unfavorable interactions. These features include the introduction of a previously unseen complementary charged residue mesh on β-sheet 2, which we propose to contribute to the slow unfolding rate. FN3con includes the optimization of alignment within the hydrophobic core, resulting in superior packing, followed by removal of solvent exposed hydrophobic residues and widespread adoption of tyrosine residues. Dynamics simulations reinforce the stability hierarchy determined by experiment and shed light on behavior of the FN3 domain at high temperature. Furthermore, we are the first to suggest liability of the N and C terminal strands of the FN3 domain via strand swapping to be part of the unfolding pathway at high temperature; we attribute this to stability by the ability to dynamically adapt hydrophobic residues during conformational change. As such, FN3con is capable of near perfect realignment of the hydrophobic core and recruitment of tyrosine residues as required. By exploiting the increased availability of genomic sequence data, this study further supports consensus design to be a rapid and effective method for the engineering of protein stability.

### Example 5: Rational Loop Grafting on FN3con

The three binding loops (B/C, D/E and F/G) from a FN3 domain that has previously been evolved to bind lysozyme at 1 pM affinity were grafted onto the FN3con scaffold. The loops were originally evolved on the FNfn10 scaffold (Hackel et al., 2008) in the construct termed DE0.4.1. The evolved construct has a significantly reduced stability, is highly aggregation prone and expresses insolubly.

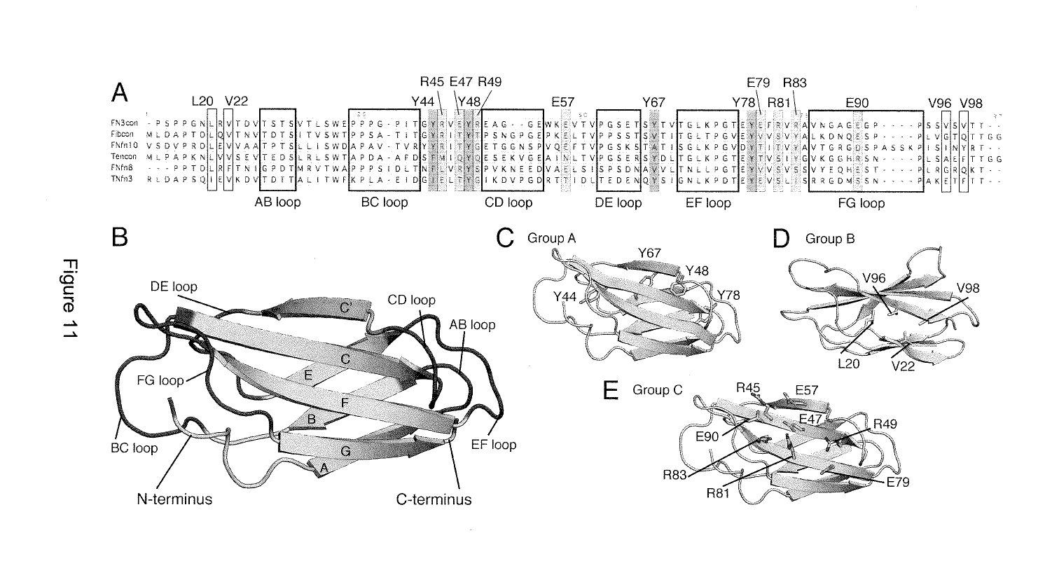

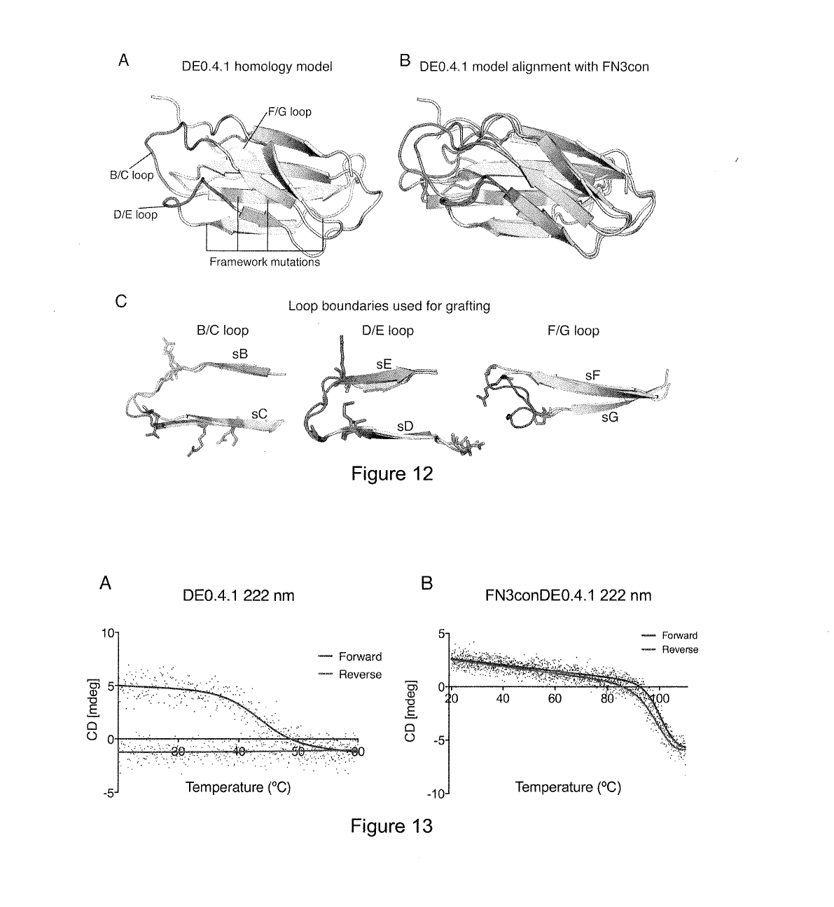

The loops from DE0.4.1 were rationally grafted by creating a homology model of DE0.4.1 and alignment with the FN3con scaffold (FIGS. 12 A, B and C). From this homology model, loop boundaries were predicted and the graft was made in silico. The designed graft (FN3con.DE0.4.1_graft_v1) was created by gene synthesis, protein expression in E. coli and subsequent purification. We found the graft to have lost some stability in comparison to the scaffold, but a melting temperature of 100° C. was observed, along with full reversible folding and soluble protein expression (FIG. 13 A and FIG. 13 B).

The biophysical characterization reveals that the FN3con scaffold better tolerates the DE0.4.1 loops better than the FNfn10 scaffold, predicting superiority for evolving loops with greater sequence space availability. However, the characterization revealed an incompatibility of the DE0.4.1 loops on the FN3con scaffold with a substantial reduction in binding affinity 1 μM to −100 μM (data not shown).

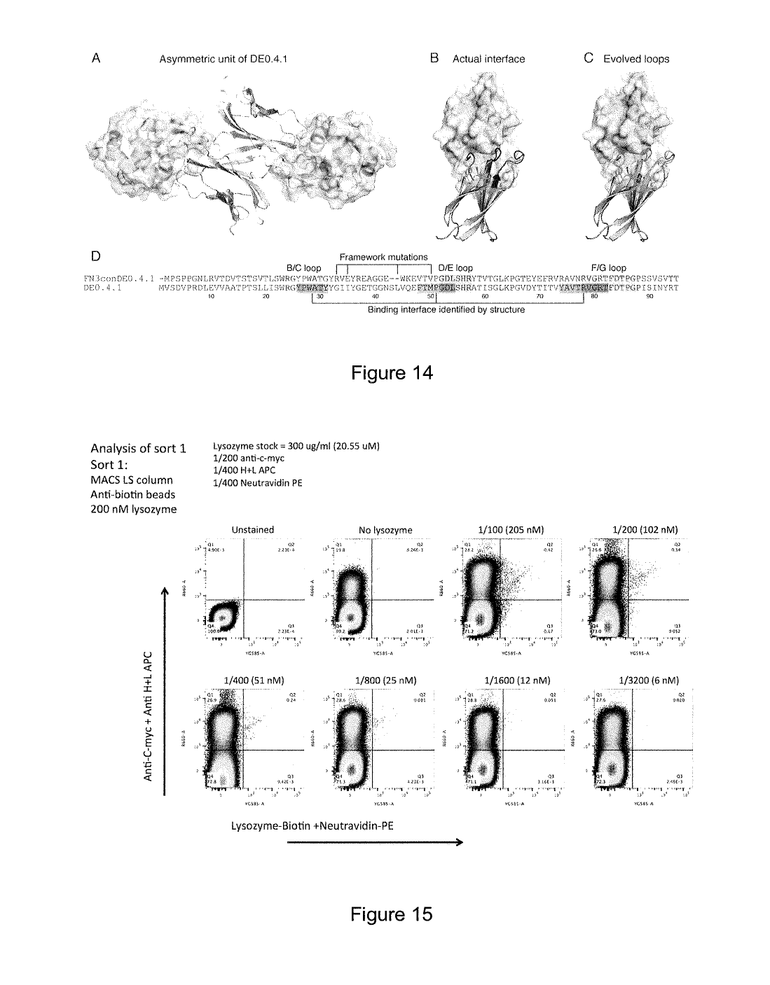

In A recently deter fined crystal structure of DE0.4.1 in complex with lysozyme revealed that DE0.4.1 was evolved to use parts of the framework region in binding that are not complementary with the FN3con scaffold. A redesign of DE0.4.1 with the FN3con scaffold was undertaken (see FIGS. 14 A, B and C).

Regions in the FN3con scaffold that were incompatible with the lysozyme interface were identified. The FN3con graft was mutated to make the interface as similar as possible to DE0.4.1. The mutations were:

G30Y, R32G, E44Q, T46F, V47T, insertion (M between 47 and 48), R71Y.
This produces the following sequences:

>FN3con_DE0.4.1_graft_v1
(SEQ ID NO: 12)
PSPPGNLRVTDVTSTSVTLSWRGYPWATGYRVEYREAGGEWKEVTV-
PGDLSHRYTVTGLKPGTEYEFRVRAVNRVGRTFDTPGPSSVSVTT
>FN3con DE0.4.1_graft_v2
(SEQ ID NO: 13)
PSPPGNLRVTDVTSTSVTLSWRGYPWATYYGVEYREAGGEWKQVFTMPGD
LSHRYTVTGLKPGTEYEFRVYAVNRVGRTFDTPGPSSVSVTT

The reasoning for these mutations can be seen in FIGS. 14 A, B and C. The crystal structure of DE0.4.1 in complex with lysozyme reveals that the binding interface utilised residues outside of the evolved loops. This was not anticipated in the original design of the graft. The redesign attempts to mimic the DE0.4.1 binding interface in the FN3con scaffold to overcome the reduced binding affinity seen with FN3con.DE0.4.1_graft_v1. It is expected that FN3con_DE0.4.1_graft_v2 will have binding affinity of at least low nM and most likely low pM.

### Example 6: Design and Implementation of a Yeast Surface Display Library

In this stream of work the inventors took their FN3con scaffold and structurally defined loop boundaries based on what has been conducted in the literature, and what they knew about the key stabilizing regions of FN3con. The library comprises an intentional insertion between residues 57 and Y68 because that loop in FN3con was potentially too short for binding. The boundaries encompass the B/C, D/E and F/G loops and replace each codon with the degenerate NNS codon (listed as X in the protein sequence).

>FN3con.NNS.library
(SEQ ID NO: 11)
MPSPPGNLRVTDVTSTSVTLSWEXXXXXXXGYRVEYREAGGEWKEVTVPX
XXXXSYTVTGLKPGTEYEFRVRAXXXXXXXXPSSVSVTT

The library was synthesized by Genscript as individual loop cassettes that were assembled by homologous recombination in yeast cells (EBY100), along with the yeast surface display vector pCTcon2. Each loop cassette was synthesized with approximately 10¹¹ diversity, producing a maximum library size of 3×10¹¹ once assembled. The transformation efficiency of yeast is approximately 1×10⁹ with the protocols used, thus producing a library with diversity somewhere between 1×10⁸ and 1×10⁹.

The library was sorted for binders against lysozyme. The procedure involved an initial round of magnetic bead enrichment by mixing displaying cells with 200 nM of biotinylated lysozyme with anti-biotin magnetic beads. This mixture was then flowed over a Miltenyi Biotec LS column in a magnetic field, washed and bound cells eluted. The eluted cells were proliferated and assessed for lysozyme binding by flow cytometry titration (FIG. 15). Titration reveals a significant double positive population of cells, well exceeding background levels of neutravidin-PE binding, all the way down to 6 nM of lysozyme—suggesting the capacity for the library to possess low nM binders without the need for affinity maturation.

### REFERENCES

Adams, P. D. et al. (2010) PHENIX: a comprehensive Python-based system for acromolecular structure solution. Acta Crystallogr. D Biol. Crystallogr., 66, 213-221.
Bakan, A. et al. (2011) ProDy: protein dynamics inferred from theory and experiments. Bioinformatics, 27, 1575-1577.
Battye, T. G. G. et al. (2011) iMOSFLM: a new graphical interface for diffraction image processing with MOSFLM. Acta Crystallogr. D Biol. Crystallogr., 67, 271-281.
Berendsen, H. et al. (1981) Interaction models for water in relation to protein hydration. Intermolecular forces, 11, 331-342.
Berendsen, H. J. C. et al. (1984) Molecular dynamics with coupling to an external bath. J. Chem. Phys., 81, 3684.
Clarke, J. and Fersht, A. R. (1993) Engineered disulfide bonds as probes of the folding pathway of barnase: increasing the stability of proteins against the rate of denaturation. Biochemistry, 32, 4322-4329.
Cota, E. and Clarke, J. (2000) Folding of beta-sandwich proteins: three-state transition of a fibronectin type III module. Protein Sci., 9, 112-120.
DeLano, W. L. (2002) The PyMOL Molecular Graphics System. (2002).
Dundas, J. et al. (2006) CASTp: computed atlas of surface topography of proteins with structural and topographical mapping of functionally annotated residues. Nucleic Acids Res., 34, W116-8.
Emsley, P. and Cowtan, K. (2004) Coot: model-building tools for molecular graphics. Acta Crystallogr. D Biol. Crystallogr., 60, 2126-2132.
Eswar, N. et al. (2007) Comparative protein structure modeling using MODELLER. Curr Protoc Protein Sci, Chapter 2, Unit 2.9.
Fleming, P. J. and Richards, F. M. (2000) Protein packing: dependence on protein size, secondary structure and amino acid composition. Journal of Molecular Biology, 299, 487-498.
Hamill, S. J. et al. (1998) The effect of boundary selection on the stability and folding of the third fibronectin type III domain from human tenascin. Biochemistry, 37, 8071-8079.
Hackel, B. J., Kapila, A., and Dane Wittrup, K. (2008) Picomolar Affinity Fibronectin Domains Engineered Utilizing Loop Length Diversity, Recursive Mutagenesis, and Loop Shuffling. Journal of Molecular Biology 381, 1238-1252.
Heinz, T. N. et al. (2001) Comparison of four methods to compute the dielectric permittivity of liquids from molecular dynamics simulations. J Chem. Phys., 115, 1125.
Hekkelman, M. L. et al. (2010) WIWS: a protein structure bioinformatics Web service collection. Nucleic Acids Res., 38, W719-23.
Hess, B. et al. (2008) GROMACS 4: Algorithms for Highly Efficient, Load-Balanced, and Scalable Molecular Simulation. J. Chem. Theory Comput., 4, 435-447.
Hess, B. et al. (1997) LILACS: a linear constraint solver for molecular simulations. J. Comput. Chem., 18, 1463-1472.
Huang, Y. et al. (2010) CD-HIT Suite: a web server for clustering and comparing biological sequences. Bioinformatics, 26, 680-682.
Humphrey, W. et al. (1996) VMD: visual molecular dynamics. J Mol Graph, 14, 33-8-27-8.
Hunter, J. D. (2007) Matplotlib: A 2D Graphics Environment. Comput. Sci. Eng., 9, 90-95.
Jacobs, S. A. et al. (2012) Design of novel FN3 domains with high stability by a consensus sequence approach. Protein Eng. Des. Sel., 25, 107-117.
Konagurthu, A. S. et al. (2010) MUSTANG-MR Structural Sieving Server: Applications in Protein Structural Analysis and Crystallography. PLoS ONE, 5, e10048.
Kyte, J. and Doolittle, R. F. (1982) A simple method for displaying the hydropathic character of a protein. Journal of Molecular Biology, 157, 105-132.
Larkin, M. A. et al. (2007) Clustal W and Clustal X version 2.0. Bioinformatics, 23, 2947-2948.
McCoy, A. J. et al. (2007) Phaser crystallographic software. J Appl Crystallogr, 40, 658-674.
Miyamoto, S. and Kollman, P. A. (1992) SETTLE: an analytical version of the SHAKE and RATTLE algorithm for rigid water models. J Comput. Chem., 13, 952-962.
Ng, S. P. et al. (2007) Designing an extracellular matrix protein with enhanced mechanical stability. Proc. Natl. Acad. Sci. U.S.A., 104, 9633-9637.
Oostenbrink, C. et al. (2004) A biomolecular force field based on the free enthalpy of hydration and solvation: The GROMOS force-field parameter sets 53A5 and 53A6. J. Comput. Chem., 25, 1656-1676.
Royer, C. A. et al. (1993) Resolution of the fluorescence equilibrium unfolding profile of trp aporepressor using single tryptophan mutants. Protein Sci., 2, 1844-1852.
Santoro, M. M. and Bolen, D. W. (1988) Unfolding free energy changes determined by the linear extrapolation method. 1. Unfolding of phenylmethanesulfonyl alpha-chymotrypsin using different denaturants. Biochemistry, 27, 8063-8068.
Tironi, I. G. et al. (1995) A generalized reaction field method for molecular dynamics simulations. J. Chem. Phys., 102, 5451.
Winn, M. D. et al. (2011) Overview of the CCP4 suite and current developments. Acta Crystallogr. D Biol. Crystallogr., 67, 235-242.

1: A highly stable polypeptide scaffold comprising an amino acid sequence based on a fibronectin type III (Fn3) domain, in which the amino acid sequence comprises at least:
(A) Y44, Y48, Y67 and Y78;
(B) L20 and V96 and/or V22 and V98; and
(C) R45 and one, two, three, four, five or all of R49, R81, R83, E47, E57 and E79, wherein amino acids are numbered according to their position in SEQ ID NO: 1.
(SEQ ID NO: 1)
X₁₋₁₃ XXXXXX LXVXXXXXXX XXXXXXXXXX XXXXYRXEYR
XXXXXXXEXX XXXXXXXYXX XXXXXXXXYE XRXRXXXXXX
EXXXXXVXVXX

2: The polypeptide scaffold of claim 1 further comprising E90.

3: The polypeptide scaffold of claim 1 in which the amino acid residues of Group A protect a hydrophobic core via mediation of interactions with water.

4: The polypeptide scaffold of claim 1 in which the amino acid residues of Group B provide favourable interactions amongst the N and C terminal strands of the molecule at elevated temperatures.

5: The polypeptide scaffold of claim 1 in which the amino acid residues of Group C provide a complementary salt bridge network on the surface of β-sheet 2 which restricts unfolding of the molecule at high temperatures.

6: The polypeptide scaffold of claim 1 comprising the amino acid sequence of residues 14-98 of SEQ ID NO: 1, in which X represents any amino acid residue.
(SEQ ID NO: 1)
X₁₋₁₃ XXXXXX LXVXXXXXXX XXXXXXXXXX XXXXYRXEYR
XXXXXXXEXX XXXXXXXYXX XXXXXXXXYE XRXRXXXXXX
EXXXXXVXVXX

7: The polypeptide scaffold of claim 6 comprising the amino acid sequence of residues 14-98 of SEQ ID NO: 2 or a variant thereof, with non-essential residues shown in lower case.
X₁₋₁₃psppgn LrVtdvtsts vtlswepppg pitgYRvEYR eaggewkEvt vpgsetsYtv tglkpgteYE fRvRavngag EgppssVsVtt (SEQ ID NO:2).

8: A polypeptide comprising the polypeptide scaffold of claim 1 incorporating one or more insertions or substitutions in one or more loop regions of the scaffold.

9: An isolated nucleic acid molecule encoding the polypeptide scaffold of claim 1 or complementary to the nucleic acid molecule encoding the polypeptide scaffold of claim 1 or capable of hybridising to the nucleic acid molecule encoding the polypeptide scaffold of claim 1 under selected stringency conditions.

10: A vector comprising the nucleic acid molecule of claim 9.

11: A host cell comprising and optionally transformed with the nucleic acid molecule of the claim 9.

12: A method of making the stable polypeptide scaffold of claim 1, the method comprising culturing a host cell to produce the polypeptide scaffold or polypeptide and recovering the scaffold or polypeptide, wherein the host cell comprises and optionally is transformed with a nucleic acid molecule encoding the polypeptide scaffold of claim 1 or complementary to the nucleic acid molecule encoding the polypeptide scaffold of claim 1 or capable of hybridising to the nucleic acid molecule encoding the polypeptide scaffold of claim 1 under selected stringency conditions.

13: A polypeptide scaffold or polypeptide produced by the method of claim 12.

14: A method of generating a library of the polypeptide scaffolds of claim 1, the method comprising incorporating randomised codons in order to produce polypeptide variants, comprising the steps of introducing randomised codons into a nucleic acid molecule at selected positions, wherein the nucleic acid molecule encodes the polypeptide scaffold of claim 1 or is complementary to the nucleic acid molecule encoding the polypeptide scaffold of claim 1 or is capable of hybridising to the nucleic acid molecule encoding the polypeptide scaffold of claim 1 under selected stringency conditions, and propagating copies of the nucleic acid molecule to form a library of nucleic acid molecules encoding variant scaffold proteins.

15: A scaffold library comprising the polypeptide scaffold of claim 1.

16: A method of generating a scaffold molecule that binds to a particular target by panning the scaffold library of claim 15 with the target and detecting binders.

17: An isolated nucleic acid molecule encoding the polypeptide of claim 8 or complementary to the nucleic acid molecule encoding the polypeptide of claim 8 or capable of hybridising to the nucleic acid molecule encoding the polypeptide of claim 8 under selected stringency conditions.

18: A scaffold library generated by the method of claim 14.

19: A method of generating a scaffold molecule that binds to a particular target by panning the scaffold library of claim 18 with the target and detecting binders.

20: A host cell comprising and optionally transformed with the vector of claim 10.


SEQUENCE LISTING (13 sequences)

SEQ ID NO: 1 - protein, 100 aa, Artificial
  Synthetic sequence
  misc_feature (1)..(19): Xaa can be any naturally occurring amino acid
  misc_feature (21)..(21): Xaa can be any naturally occurring amino acid
  misc_feature (23)..(43): Xaa can be any naturally occurring amino acid
  misc_feature (46)..(46): Xaa can be any naturally occurring amino acid
  misc_feature (50)..(56): Xaa can be any naturally occurring amino acid
  misc_feature (58)..(66): Xaa can be any naturally occurring amino acid
  misc_feature (68)..(77): Xaa can be any naturally occurring amino acid
  misc_feature (80)..(80): Xaa can be any naturally occurring amino acid
  misc_feature (82)..(82): Xaa can be any naturally occurring amino acid
  misc_feature (84)..(89): Xaa can be any naturally occurring amino acid
  misc_feature (91)..(95): Xaa can be any naturally occurring amino acid
  misc_feature (97)..(97): Xaa can be any naturally occurring amino acid
  misc_feature (99)..(100): Xaa can be any naturally occurring amino acid
XXXXXXXXXXXXXXXXXXXLXVXXXXXXXXXXXXXXXXXXXXXYRXEYRXXXXXXXEXXXXXXXXXYXXXXXXXXXXYEXRXRXXXXXXEXXXXXVXVXX

SEQ ID NO: 2 - protein, 100 aa, Artificial
  Synthetic sequence
  misc_feature (1)..(13): Xaa can be any naturally occurring amino acid
XXXXXXXXXXXXXPSPPGNLRVTDVTSTSVTLSWEPPPGPITGYRVEYREAGGEWKEVTVPGSETSYTVTGLKPGTEYEFRVRAVNGAGEGPPSSVSVTT

SEQ ID NO: 3 - protein, 101 aa, Homo sapiens
VSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQ

SEQ ID NO: 4 - protein, 93 aa, Homo sapiens
PLSPPTNLHLEANPDTGVLTVSWERSTTPDITGYRITTTPTNGQQGNSLEEVVHADQSSCTFDNLSPGLEYNVSVYTVKDDKESVPISDTIIP

SEQ ID NO: 5 - protein, 89 aa, Homo sapiens
NVSPPRRARVTDATETTITISWRTKTETITGFQVDAVPANGQTPIQRTIKPDVRSYTITGLQPGTDYKIYLYTLNDNARSSPVVIDAST

SEQ ID NO: 6 - protein, 90 aa, Artificial
  Synthetic sequence
RLDAPSQIEVKDVTDTTALITWFKPLAEIDGIELTYGIKDVPGDRTTIDLTEDENQYSIGNLKPDTEYEVSLISRRGDMSSNPAKETFTT

SEQ ID NO: 7 - protein, 88 aa, Artificial
  Synthetic sequence
PPTDLRFTNIGPDTMRVTWAPPPSIDLTNFLVRYSPVKNEEDVAELSISPSDNAVVLTNLLPGTEYVVSVSSVYEQHESTPLRGRQKT

SEQ ID NO: 8 - protein, 94 aa, Artificial
  Synthetic sequence
VSDVPRDLEVVAATPTSLLISWDAPAVTVRYYRITYGETGGNSPVQEFTVPGSKSTATISGLKPGVDYTITVYAVTGRGDSPASSKPISINYRT

SEQ ID NO: 9 - protein, 92 aa, Artificial
  Synthetic sequence
MLPAPKNLVVSEVTEDSLRLSWTAPDAAFDSFMIQYQESEKVGEAINLTVPGSERSYDLTGLKPGTEYTVSIYGVKGGHRSNPLSAEFTTGG

SEQ ID NO: 10 - protein, 92 aa, Artificial
  Synthetic sequence
MLDAPTDLQVTNVTDTSITVSWTPPSATITGYRITYTPSNGPGEPKELTVPPSSTSVTITGLTPGVEYVVSVYALKDNQESPPLVGTQTTGG

SEQ ID NO: 11 - protein, 89 aa, Artificial
  Synthetic sequence
  misc_feature (24)..(30): Xaa can be any naturally occurring amino acid
  misc_feature (50)..(54): Xaa can be any naturally occurring amino acid
  misc_feature (74)..(81): Xaa can be any naturally occurring amino acid
MPSPPGNLRVTDVTSTSVTLSWEXXXXXXXGYRVEYREAGGEWKEVTVPXXXXXSYTVTGLKPGTEYEFRVRAXXXXXXXXPSSVSVTT

SEQ ID NO: 12 - protein, 91 aa, Artificial
  Synthetic sequence
PSPPGNLRVTDVTSTSVTLSWRGYPWATGYRVEYREAGGEWKEVTVPGDLSHRYTVTGLKPGTEYEFRVRAVNRVGRTFDTPGPSSVSVTT

SEQ ID NO: 13 - protein, 92 aa, Artificial
  Synthetic sequence
PSPPGNLRVTDVTSTSVTLSWRGYPWATYYGVEYREAGGEWKQVFTMPGDLSHRYTVTGLKPGTEYEFRVYAVNRVGRTFDTPGPSSVSVTT